(19) United States
(12) Patent Application Publication
(10) Pub. No.: US 2017/0182283 A1
(43) Pub. Date: Jun. 29, 2017

(54) METHODS AND SYSTEMS FOR ENHANCING SLEEP OF A USER OF AN INTERACTIVE MEDIA GUIDANCE SYSTEM

(71) Applicant: Rovi Guides, Inc., Santa Clara, CA (US)

(72) Inventors: Lauren Palmateer, San Francisco, CA (US); Walter R. Klappert, Los Angeles, CA (US)

(21) Appl. No.: 14/998,080

(22) Filed: Dec. 23, 2015

(51) Int. Cl. A61M 21/02; A61B 5/021; A61B 5/024; G06F 19/00; A61B 5/00

(52) U.S. Cl. CPC A61M 21/02; G06F 19/3406; A61B 5/4809; A61B 5/4812; A61B 5/0077; A61B 5/024; A61B 5/021; A61B 5/443; A61B 5/4064; A61B 5/7246; A61M 2021/0027

(57) ABSTRACT

Methods and systems for enhancing sleep of a user. In some aspects, control circuitry receives a first input relating to a first sleep state. The control circuitry determines whether the first sleep state indicates the user has fallen asleep. In response to determining the user has fallen asleep, the control circuitry determines a first media asset corresponding to the first sleep state. The control circuitry generates for display the first media asset to enhance the user's sleep. The control circuitry receives a second input relating to a second sleep state. The control circuitry determines whether the second sleep state indicates the user is ready to be awakened. In response to determining the user is ready to be awakened, the control circuitry determines a second media asset corresponding to the second sleep state. The control circuitry generates for display the second media asset to awaken the user.

### BACKGROUND OF THE INVENTION

In conventional systems, a user can set a timer to turn off or turn on a television display. For example, the user may set a turn-off timer for one hour while he or she is trying to fall asleep at night. However, the television display may interfere with the user's ability to fall asleep or turn off before the user has fallen asleep or otherwise interfere with the user's sleep. In another example, the user may set a turn-on timer for eight hours to wake the user up. However, the television display may cause anxiety to the user, for example, if a loud commercial plays, when the television display is turned on. Conventional systems lack the ability to address such issues with the user's sleep.

### SUMMARY

Accordingly, methods and systems are disclosed herein for enhancing the sleep of a user of an interactive media guidance system. Sound and light, alone or in combination, can alter people's sleep states and enhance the sleeping experience when they sleep. In one example, the system provides a user an option for alert dozing that can be accompanied by educational audio and/or images. Language-learning while dozing is a well-known example of this learning technique. In another example, the system provides the user an option for deep sleep in which the system turns off all sound and darkens its display. The system may turn on a noise cancellation feature if the user is wearing earphones or headphones or another suitable headset. The user may vary his or her nap profile according to the time of day, the day of the week, or other factors. For example, the system may automatically initiate a set of sleep-enhancing features at midnight while initiating a different set of sleep-enhancing features just after lunchtime.

In some embodiments, the interactive media guidance system includes control circuitry for implementing an interactive media guidance application. The interactive media guidance application may include functionality to enhance sleep of the user. For example, the interactive media guidance application may take steps to enhance sleep based on a position of the user, a sound from or near the user, a biometric reading from the user, or other suitable methods for determining the user's sleep state such as wireless x-ray vision and detection of eyelids and rapid eye movement. The interactive media guidance system may include one or more detection devices, such as a microphone, a camera, a smart watch, a biometric sensor, an eye movement sensor, or another suitable detection device, to gather relevant information for the user and his or her sleep state.

In some embodiments, the interactive media guidance application receives measurements from the detection device relating to the user's sleep state. For example, the interactive media guidance application may receive a position of the user from a camera (for example, sitting up indicating awake or lying down indicating asleep), a sound from or near the user from a microphone (for example, cheering sounds indicating awake or light snoring indicating asleep), a heart rate from a biometric sensor on the user (for example, a higher heart rate indicating awake or a lower heart rate indicating asleep), or another suitable measurement relating to the user's sleep state. One or more of these measurements may be included in a data structure representing the user's sleep state. The sleep state indicates the current state of the user, for example, whether the user is awake, whether the user is asleep, or another suitable state of the user.

In another example, the interactive media guidance application may receive brain activity data from a detection device, such as an electroencephalographic (EEG) headset. Sleep is typically broken into several stages that the brain cycles through approximately every 90 to 120 minutes. These stages are broadly characterized into non-rapid eye movement (NREM) sleep and rapid eye movement (REM) sleep. NREM is further broken down into stage one and two, which are considered light and intermediate sleep, followed by slow-wave sleep. Awakening from slow-wave sleep may result in sleep inertia or sleep drunkenness, a groggy feeling that can take awhile to shake off. Finally, REM sleep is often associated with dreaming. The EEG headset may monitor brain activity and determine the sleep stage the user is currently experiencing and store the information as part of the user's sleep state. In some embodiments, the sleep state indicates one of several states of the user. For example, the sleep state may indicate whether the user is dreaming (for example, when the user is experiencing REM sleep), whether the user is in deep sleep (for example, when the user is experiencing slow-wave sleep), whether the user is alert dozing (for example, when the user is experiencing NREM sleep), whether the user is sleepy (for example, when the user's heart rate is below a certain threshold), whether the user is awake (for example, when the user's heart rate is above a certain threshold), or another suitable state of the user.

In some embodiments, the interactive media guidance application detects that the user has fallen asleep based on the user's sleep state. For example, when the interactive media guidance application detects that the user has fallen asleep, the interactive media guidance application may turn off the television display. In another example, the interactive media guidance application may instruct a speaker to produce certain sounds, such as drumming music, to help soothe the user during sleep. In yet another example, the interactive media guidance application may instruct a display to produce certain types of light, such as soft green light, to help soothe the user during sleep. In yet another example, the interactive media guidance application may initiate subliminal learning by playing an audio lesson for a foreign language the user wishes to learn.

In some embodiments, the interactive media guidance application detects that the user is ready to be awakened based on the user's sleep state. The interactive media guidance application may wake the user at the proper time for the desired state of consciousness. For example, the interactive media guidance application may detect the user's brain activity and avoid waking the user during slow-wave sleep to avoid grogginess due to sleep inertia. In some embodiments, the interactive media guidance application presents options for viewing what the user was viewing before he or she fell asleep.

In some embodiments, the interactive media guidance application provides options for an enhanced nap to the user. The user may choose to take a nap and doze with soothing sounds and light or be escorted into a more alert state and have, for example, a French lesson, played during his or her nap for the desired outcome of the user becoming more proficient in the French language. Additionally and optionally, the interactive media guidance application may select specific information to be conveyed during specific sleep states that are conducive to learning. It is well known that different states of consciousness associated with the various sleep states have varying degrees of susceptibility in human beings. The interactive media guidance application may promote the desired information personalized to the user as he or she is going into a napping state to promote the desired effect or communicate the desired information.

In some embodiments, the interactive media guidance application automatically sets an alarm to awaken the user after a specific length of time. The user may set how long his or her nap should be based on the time, day, or other conditions when he or she falls asleep. The interactive media guidance application may use an electrical signal, for example, generated via a smart watch worn by the user, to gently wake the user and avoid grogginess due to sleep inertia. The interactive media guidance application may provide selectable options to the user to wake to an audio or video asset, an audio alert, an electrical signal from a biometric device worn by the user, or another suitable alert.

In some embodiments, the interactive media guidance application allows the user to catch up on media assets the user missed while he or she was sleeping. The interactive media guidance application may detect that the user has fallen asleep and initiate recording of the media asset currently being viewed. When the interactive media guidance application wakes up the user, the interactive media guidance application may offer selectable options to provide a summary of the portion of the media asset the user missed, to play back the recording of the portion of the media asset the user missed, or to play back only important scenes from the portion of the media asset the user missed. The interactive media guidance application may set an alarm for an important scene that the user may not want to miss. Additionally and optionally, the interactive media guidance application may allow the user to choose how long he or she should be allowed to nap before he or she is awakened. The interactive media guidance application may set an automatic alarm to awaken the user accordingly.

In an illustrative example, Albert makes the most of his habit of drifting off to sleep around 4:00 PM on Saturday and Sunday afternoons in front of his television display. In the interactive media guidance application, Albert opts for a napping profile to help him learn spoken Mandarin Chinese. Using detection devices, including a camera and biometric sensors to provide heartbeat, blood pressure, skin moisture readings, and rapid eye movement, the interactive media guidance application detects when Albert falls asleep. If it is a weekend afternoon, the interactive media guidance application plays audio conversations in Mandarin Chinese at Albert's appropriate difficulty level for 20 minutes. After 20 minutes, the interactive media guidance application raises the audio speaker volume and creates a buzzing sound accompanied by flashing bright colors on the television display to wake Albert up as he requested in his profile. Albert sometimes also uses an EEG headband that provides the interactive media guidance application data regarding Albert's sleep levels. In such cases, the interactive media guidance application uses the EEG headband data to determine when to wake Albert to avoid grogginess due to sleep inertia.

In some aspects, the systems and methods described herein provide for enhancing sleep of a user. The described systems and methods may be implemented in an interactive media guidance application. The interactive media guidance application receives, from a detection device, a first input relating to a first sleep state of the user. For example, the first sleep state may include measurements from one or more detection devices, such as a video camera, a microphone, and a biometric sensor. The interactive media guidance application determines whether the first sleep state indicates that the user has fallen asleep. For example, the first sleep state may include measurements relating to the user that indicate the user has fallen asleep, such as the user may be lying down, may be lightly snoring, and/or may have a lower heart rate.

In response to determining that the first sleep state indicates that the user has fallen asleep, the interactive media guidance application determines a first media asset corresponding to the first sleep state of the user. The first media asset enhances the user's sleep. For example, the interactive media guidance application may select a media asset, including soothing sounds and light to help the user fall into deep sleep. In another example, the interactive media guidance application may select a media asset for an audio foreign language lesson to help the user learn the foreign language. The interactive media guidance application generates for aural or visual display the first media asset to enhance the user's sleep.

The interactive media guidance application receives, from the detection device, a second input relating to a second sleep state of the user. For example, the second sleep state may include measurements from one or more detection devices, such as a video camera, a microphone, and a biometric sensor. The interactive media guidance application determines whether the second sleep state indicates that the user is ready to be awakened. For example, the second sleep state may include measurements relating to the user that indicate the user is ready to be awakened, such as the user may be moving while sleeping, may not be snoring, and/or may have a higher heart rate.

In response to determining that the second sleep state indicates that the user is ready to be awakened, the interactive media guidance application determines a second media asset corresponding to the second sleep state of the user. The second media asset wakes up the user. For example, the interactive media guidance application may select a media asset, including gradually increasing sounds and light, to help the user awaken. In another example, the interactive media guidance application may stop playback of a media asset for an audio foreign language lesson to help the user awaken. The interactive media guidance application generates for aural or visual display the second media asset to awaken the user.

In some embodiments, the detection device includes one or more of a video camera, a microphone, and a biometric sensor. For example, the interactive media guidance application may receive data from one or more detection devices, such as a microphone, a camera, a smart watch, a biometric sensor, an eye movement sensor, or another suitable detection device, to gather relevant information for the user and his or her sleep state.

In some embodiments, the first sleep state and the second sleep state comprise measurements of one or more of heartbeat, blood pressure, skin moisture, brain activity, rapid eye movement, and other suitable measurements. For example, the interactive media guidance application may receive a position of the user from a camera (for example, sitting up indicating awake or lying down indicating asleep), a sound from or near the user from a microphone (for example, cheering sounds indicating awake or light snoring indicating asleep), a heart rate from a biometric sensor on the user (for example, a higher heart rate indicating awake or a lower heart rate indicating asleep), or another suitable measurement relating to the user's sleep state. One or more of these measurements may be included in a data structure representing the user's sleep state.

In some embodiments, determining whether the first sleep state indicates that the user has fallen asleep comprises comparing the first sleep state to a first reference sleep state indicative of a reference user who has fallen asleep and determining whether the first sleep state substantially matches the first reference sleep state. For example, measurements from one or more detection devices may be included in a data structure representing the first sleep state. The interactive media guidance application may compare the first sleep state to the reference sleep state and determine whether the corresponding measurements differ within or beyond a threshold.

In some embodiments, determining the first media asset corresponding to the first sleep state of the user comprises retrieving, from memory, one or more media assets specified by the user for enhancing the user's sleep and selecting the first media asset from the one or more media assets. For example, the user may have selected an audio foreign language lesson to be played back when the user falls asleep.

In some embodiments, determining the first media asset corresponding to the first sleep state of the user comprises retrieving, from memory, a user profile for the user, searching a database for one or more media assets for enhancing the user's sleep based on the user profile, and automatically selecting the first media asset from the one or more media assets. For example, the user may have been listening to an audio foreign language lesson when he or she fell asleep. The interactive media guidance application may select the next audio lesson in the series when the user falls asleep to help the user learn the foreign language.

In some embodiments, the first media asset and the second media asset comprise one of an audio asset, a video asset, and an audiovisual asset. For example, the interactive media guidance application may select an audio asset including drumming music, an audio foreign language lesson, a media asset including soft green light, a media asset including soothing sounds and light, or another suitable media asset.

In some embodiments, determining whether the second sleep state indicates the user is ready to be awakened comprises comparing the first sleep state to a second reference sleep state indicative of a reference user who is ready to be awakened and determining whether the first sleep state substantially matches the second reference sleep state. For example, measurements from one or more detection devices may be included in a data structure representing the second sleep state. The interactive media guidance application may compare the second sleep state to the reference sleep state and determine whether the corresponding measurements differ within or beyond a threshold. In another example, the interactive media guidance application may compare corresponding measurements from the received sleep state and reference sleep state, e.g., whether the user's current heartbeat of 52 beats per minute differs, within or beyond a threshold, from the reference heartbeat of 60 beats per minute, as well as check other conditions, for example, the user requested to be awakened if he is asleep at 4:30 PM. In this example, comparing the sleep states requires two comparisons, one comparison for a heartbeat of less than 60 beats per minute and another comparison to find out if the time of day is before 4:30 PM.

In some embodiments, determining the second media asset corresponding to the second sleep state of the user comprises retrieving, from memory, one or more media assets specified by the user for waking up the user and selecting the second media asset from the one or more media assets. For example, the interactive media guidance application may select a media asset including gradually increasing sounds and light to help the user awaken. In another example, the interactive media guidance application may select no media asset or a blank media asset, for example, to stop playback of a media asset for an audio foreign language lesson, to help the user awaken.

In some embodiments, determining the second media asset corresponding to the second sleep state of the user comprises retrieving, from memory, a user profile for the user, searching a database for one or more media assets for waking up the user based on the user profile, and automatically selecting the second media asset from the one or more media assets. For example, the interactive media guidance application may select a media asset including drumming sounds based on the user profile indicating that the user likes waking up to such sounds.

It should be noted that the systems, methods, apparatuses, and/or aspects described above may be applied to, or used in accordance with, other systems, methods, apparatuses, and/or aspects.

### BRIEF DESCRIPTION OF THE DRAWINGS

The above and other objects and advantages of the disclosure will be apparent upon consideration of the following detailed description, taken in conjunction with the accompanying drawings, in which like reference characters refer to like parts throughout, and in which:

FIG. 1 shows an illustrative example of a display screen generated by a media guidance application in accordance with some embodiments of the disclosure;

FIG. 2 shows another illustrative example of a display screen generated by a media guidance application in accordance with some embodiments of the disclosure;

FIG. 3 is a block diagram of an illustrative user equipment device in accordance with some embodiments of the disclosure;

FIG. 4 is a block diagram of an illustrative media system in accordance with some embodiments of the disclosure;

FIG. 5 is a flowchart of illustrative steps for determining whether the first sleep state indicates the user has fallen asleep in accordance with some embodiments of the disclosure;

FIG. 6 is pseudocode of illustrative steps for determining whether the first sleep state indicates the user has fallen asleep in accordance with some embodiments of the disclosure;

FIG. 7 is a flowchart of an illustrative process for determining whether the second sleep state indicates the user is ready to be awakened in accordance with some embodiments of the disclosure;

FIG. 8 is pseudocode of an illustrative process for determining whether the second sleep state indicates the user is ready to be awakened in accordance with some embodiments of the disclosure;

FIG. 9 is a flowchart of an illustrative process for using a database to determine a media asset corresponding to one or more sleep states of the user in accordance with some embodiments of the disclosure;

FIG. 10 is pseudocode of an illustrative process for using a database to determine a media asset corresponding to one or more sleep states of the user in accordance with some embodiments of the disclosure;

FIG. 11 is a flowchart of an illustrative process for enhancing sleep of a user of an interactive media guidance system in accordance with some embodiments of the disclosure;

FIG. 12 is a diagram of a display relating to enhancing sleep of a user of an interactive media guidance system in accordance with some embodiments of the disclosure; and

FIG. 13 is a diagram of another display relating to enhancing sleep of a user of an interactive media guidance system in accordance with some embodiments of the disclosure.

### DETAILED DESCRIPTION

Methods and systems for enhancing sleep of a user of an interactive media guidance system are described. The interactive media guidance system may include control circuitry for implementing an interactive media guidance application. The interactive media guidance application may include functionality to enhance sleep of the user.

In some aspects, the control circuitry receives a first input relating to a first sleep state of the user. The control circuitry determines whether the first sleep state indicates that the user has fallen asleep. In response to determining that the first sleep state indicates that the user has fallen asleep, the control circuitry determines a first media asset corresponding to the first sleep state of the user. The control circuitry generates for display the first media asset to enhance the user's sleep.

The control circuitry receives a second input relating to a second sleep state of the user. The control circuitry determines whether the second sleep state indicates that the user is ready to be awakened. In response to determining that the second sleep state indicates that the user is ready to be awakened, the control circuitry determines a second media asset corresponding to the second sleep state of the user. The control circuitry generates for display the second media asset to awaken the user.

The amount of content available to users in any given content delivery system can be substantial. Consequently, many users desire a form of media guidance through an interface that allows users to efficiently navigate content selections and easily identify content that they may desire. An application that provides such guidance is referred to herein as an interactive media guidance application or, sometimes, a media guidance application or a guidance application.

Interactive media guidance applications may take various forms depending on the content for which they provide guidance. One typical type of media guidance application is an interactive television program guide. Interactive television program guides (sometimes referred to as electronic program guides) are well-known guidance applications that, among other things, allow users to navigate among and locate many types of content or media assets. Interactive media guidance applications may generate graphical user interface screens that enable a user to navigate among, locate and select content. As referred to herein, the terms “media asset” and “content” should be understood to mean an electronically consumable user asset, such as television programming, as well as pay-per-view programs, on-demand programs (as in video-on-demand (VOD) systems), Internet content (for example, streaming content, downloadable content, Webcasts, etc.), video clips, audio, content information, pictures, rotating images, documents, playlists, websites, articles, books, electronic books, blogs, advertisements, chat sessions, social media, applications, games, and/or any other media or multimedia and/or combination of the same. Guidance applications also allow users to navigate among and locate content. As referred to herein, the term “multimedia” should be understood to mean content that utilizes at least two different content forms described above, for example, text, audio, images, video, or interactivity content forms. Content may be recorded, played, displayed or accessed by user equipment devices, but can also be part of a live performance.

The media guidance application and/or any instructions for performing any of the embodiments discussed herein may be encoded on computer readable media. Computer readable media includes any media capable of storing data: The computer readable media may be transitory, including, but not limited to, propagating electrical or electromagnetic signals, or may be non-transitory including, but not limited to, volatile and non-volatile computer memory or storage devices such as a hard disk, floppy disk, USB drive, DVD, CD, media cards, register memory, processor caches, Random Access Memory (“RAM”), etc.

With the advent of the Internet, mobile computing, and high-speed wireless networks, users are accessing media on user equipment devices on which they traditionally did not. As referred to herein, the phrase “user equipment device,” “user equipment,” “user device,” “electronic device,” “electronic equipment,” “media equipment device,” or “media device” should be understood to mean any device for accessing the content described above, such as a television, a Smart TV, a set-top box, an integrated receiver decoder (IRD) for handling satellite television, a digital storage device, a digital media receiver (DMR), a digital media adapter (DMA), a streaming media device, a DVD player, a DVD recorder, a connected DVD, a local media server, a BLU-RAY player, a BLU-RAY recorder, a personal computer (PC), a laptop computer, a tablet computer, a WebTV box, a personal computer television (PC/TV), a PC media server, a PC media center, a hand-held computer, a stationary telephone, a personal digital assistant (PDA), a mobile telephone, a portable video player, a portable music player, a portable gaming machine, a smart phone, or any other television equipment, computing equipment, or wireless device, and/or combination of the same. In some embodiments, the user equipment device may have a front facing screen and a rear facing screen, multiple front screens, or multiple angled screens. In some embodiments, the user equipment device may have a front facing camera and/or a rear facing camera. On these user equipment devices, users may be able to navigate among and locate the same content available through a television. Consequently, media guidance may be available on these devices, as well. The guidance provided may be for content available only through a television, for content available only through one or more of other types of user equipment devices, or for content available both through a television and one or more of the other types of user equipment devices. The media guidance applications may be provided as on-line applications (that is, provided on a web-site), or as stand-alone applications or clients on user equipment devices. Various devices and platforms that may implement media guidance applications are described in more detail below.

One of the functions of the media guidance application is to provide media guidance data to users. As referred to herein, the phrase “media guidance data” or “guidance data” should be understood to mean any data related to content or data used in operating the guidance application. For example, the guidance data may include program information, guidance application settings, user preferences, user profile information, media listings, media-related information (for example, broadcast times, broadcast channels, titles, descriptions, ratings information (for example, parental control ratings, critic's ratings, etc.), genre or category information, actor information, logo data for broadcasters' or providers' logos, etc.), media format (for example, standard definition, high definition, three dimensional, etc.), advertisement information (for example, text, images, media clips, etc.), on-demand information, blogs, websites, and any other type of guidance data that is helpful for a user to navigate among and locate desired content selections.

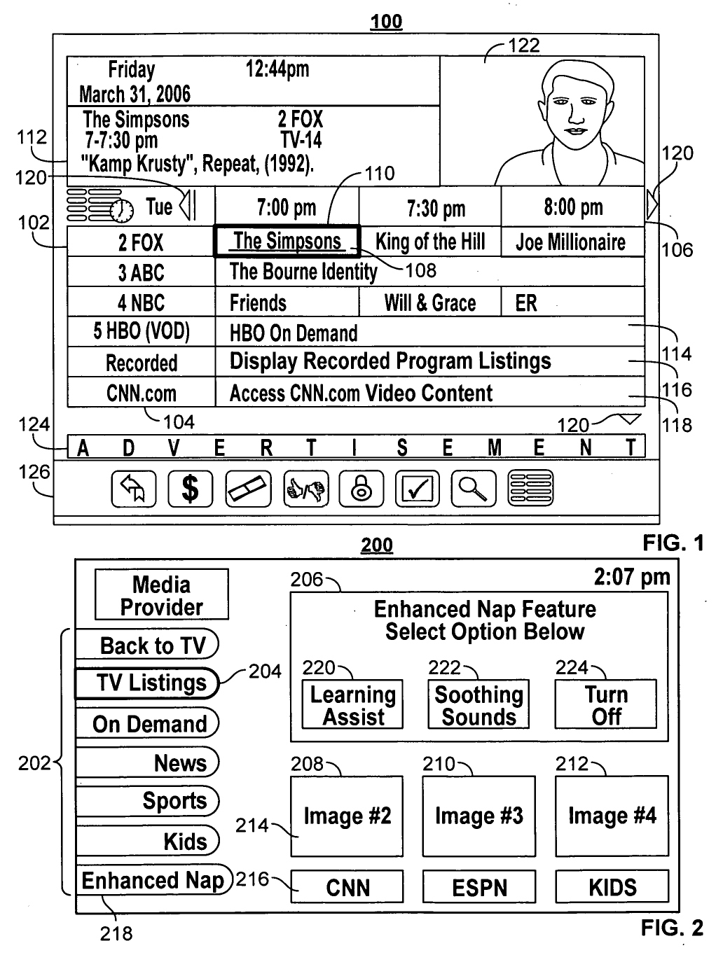

FIGS. 1-2 show illustrative display screens that may be used to provide media guidance data. The display screens shown in FIGS. 1-2 may be implemented on any suitable user equipment device or platform. While the displays of FIGS. 1-2 are illustrated as full screen displays, they may also be fully or partially overlaid over content being displayed. A user may indicate a desire to access content information by selecting a selectable option provided in a display screen (for example, a menu option, a listings option, an icon, a hyperlink, etc.) or pressing a dedicated button (for example, a GUIDE button) on a remote control or other user input interface or device. In response to the user's indication, the media guidance application may provide a display screen with media guidance data organized in one of several ways, such as by time and channel in a grid, by time, by channel, by source, by content type, by category (for example, movies, sports, news, children, or other categories of programming), or other predefined, user-defined, or other organization criteria.

FIG. 1 shows illustrative grid of a program listings display 100 arranged by time and channel that also enables access to different types of content in a single display. Display 100 may include grid 102 with: (1) a column of channel/content type identifiers 104, where each channel/content type identifier (which is a cell in the column) identifies a different channel or content type available; and (2) a row of time identifiers 106, where each time identifier (which is a cell in the row) identifies a time block of programming. Grid 102 also includes cells of program listings, such as program listing 108, where each listing provides the title of the program provided on the listing's associated channel and time. With a user input device, a user can select program listings by moving highlight region 110. Information relating to the program listing selected by highlight region 110 may be provided in program information region 112. Region 112 may include, for example, the program title, the program description, the time the program is provided (if applicable), the channel the program is on (if applicable), the program's rating, and other desired information.

In addition to providing access to linear programming (for example, content that is scheduled to be transmitted to a plurality of user equipment devices at a predetermined time and is provided according to a schedule), the media guidance application also provides access to non-linear programming (for example, content accessible to a user equipment device at any time and is not provided according to a schedule). Non-linear programming may include content from different content sources including on-demand content (for example, VOD), Internet content (for example, streaming media, downloadable media, etc.), locally stored content (for example, content stored on any user equipment device described above or other storage device), or other time-independent content. On-demand content may include movies or any other content provided by a particular content provider (for example, HBO On Demand providing “The Sopranos” and “Curb Your Enthusiasm”). HBO ON DEMAND is a service mark owned by Time Warner Company L.P. et al. and THE SOPRANOS and CURB YOUR ENTHUSIASM are trademarks owned by the Home Box Office, Inc. Internet content may include web events, such as a chat session or Webcast, or content available on-demand as streaming content or downloadable content through an Internet web site or other Internet access (for example, FTP).

Grid 102 may provide media guidance data for non-linear programming including on-demand listing 114, recorded content listing 116, and Internet content listing 118. A display combining media guidance data for content from different types of content sources is sometimes referred to as a “mixed-media” display. Various permutations of the types of media guidance data that may be displayed that are different than display 100 may be based on user selection or guidance application definition (for example, a display of only recorded and broadcast listings, only on-demand and broadcast listings, etc.). As illustrated, listings 114, 116, and 118 are shown as spanning the entire time block displayed in grid 102 to indicate that selection of these listings may provide access to a display dedicated to on-demand listings, recorded listings, or Internet listings, respectively. In some embodiments, listings for these content types may be included directly in grid 102. Additional media guidance data may be displayed in response to the user selecting one of the navigational icons 120. (Pressing an arrow key on a user input device may affect the display in a similar manner as selecting navigational icons 120.)

Display 100 may also include video region 122, advertisement 124, and options region 126. Video region 122 may allow the user to view and/or preview programs that are currently available, will be available, or were available to the user. The content of video region 122 may correspond to, or be independent from, one of the listings displayed in grid 102. Grid displays including a video region are sometimes referred to as picture-in-guide (PIG) displays. PIG displays and their functionalities are described in greater detail in Satterfield et al. U.S. Pat. No. 6,564,378, issued May 13, 2003 and Yuen et al. U.S. Pat. No. 6,239,794, issued May 29, 2001, which are hereby incorporated by reference herein in their entireties. PIG displays may be included in other media guidance application display screens of the embodiments described herein.

Advertisement 124 may provide an advertisement for content that, depending on a viewer's access rights (for example, for subscription programming), is currently available for viewing, will be available for viewing in the future, or may never become available for viewing, and may correspond to or be unrelated to one or more of the content listings in grid 102. Advertisement 124 may also be for products or services related or unrelated to the content displayed in grid 102. Advertisement 124 may be selectable and provide further information about content, provide information about a product or a service, enable purchasing of content, a product, or a service, provide content relating to the advertisement, etc. Advertisement 124 may be targeted based on a user's profile/preferences, monitored user activity, the type of display provided, or on other suitable targeted advertisement bases.

While advertisement 124 is shown as rectangular or banner shaped, advertisements may be provided in any suitable size, shape, and location in a guidance application display. For example, advertisement 124 may be provided as a rectangular shape that is horizontally adjacent to grid 102. This is sometimes referred to as a panel advertisement. In addition, advertisements may be overlaid over content or a guidance application display or embedded within a display. Advertisements may also include text, images, rotating images, video clips, or other types of content described above. Advertisements may be stored in a user equipment device having a guidance application, in a database connected to the user equipment, in a remote location (including streaming media servers), or on other storage means, or a combination of these locations. Providing advertisements in a media guidance application is discussed in greater detail in, for example, Knudson et al., U.S. Patent Application Publication No. 2003/0110499, filed Jan. 17, 2003; Ward, III et al. U.S. Pat. No. 6,756,997, issued Jun. 29, 2004; and Schein et al. U.S. Pat. No. 6,388,714, issued May 14, 2002, which are hereby incorporated by reference herein in their entireties. It will be appreciated that advertisements may be included in other media guidance application display screens of the embodiments described herein.

Options region 126 may allow the user to access different types of content, media guidance application displays, and/or media guidance application features. Options region 126 may be part of display 100 (and other display screens described herein), or may be invoked by a user by selecting an on-screen option or pressing a dedicated or assignable button on a user input device. The selectable options within options region 126 may concern features related to program listings in grid 102 or may include options available from a main menu display. Features related to program listings may include searching for other air times or ways of receiving a program, recording a program, enabling series recording of a program, setting program and/or channel as a favorite, purchasing a program, or other features. Options available from a main menu display may include search options, VOD options, parental control options, Internet options, cloud-based options, device synchronization options, second screen device options, options to access various types of media guidance data displays, options to subscribe to a premium service, options to edit a user's profile, options to access a browse overlay, or other options.

The media guidance application may be personalized based on a user's preferences. A personalized media guidance application allows a user to customize displays and features to create a personalized “experience” with the media guidance application. This personalized experience may be created by allowing a user to input these customizations and/or by the media guidance application monitoring user activity to determine various user preferences. Users may access their personalized guidance application by logging in or otherwise identifying themselves to the guidance application. Customization of the media guidance application may be made in accordance with a user profile. The customizations may include varying presentation schemes (for example, color scheme of displays, font size of text, etc.), aspects of content listings displayed (for example, only HDTV or only three dimensional programming, user-specified broadcast channels based on favorite channel selections, re-ordering the display of channels, recommended content, etc.), desired recording features (for example, recording or series recordings for particular users, recording quality, etc.), parental control settings, customized presentation of Internet content (for example, presentation of social media content, e-mail, electronically delivered articles, etc.) and other desired customizations.

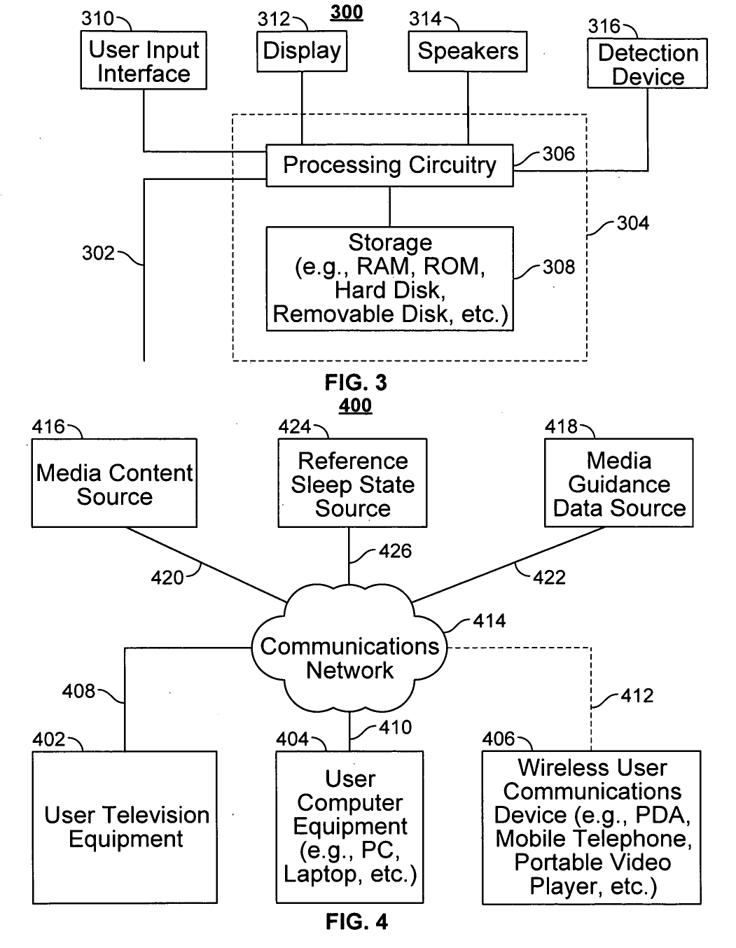

The media guidance application may allow a user to provide user profile information or may automatically compile user profile information. The media guidance application may, for example, monitor the content the user accesses and/or other interactions the user may have with the guidance application. Additionally, the media guidance application may obtain all or part of other user profiles that are related to a particular user (for example, from other web sites on the Internet the user accesses, such as www.allrovi.com, from other media guidance applications the user accesses, from other interactive applications the user accesses, from another user equipment device of the user, etc.), and/or obtain information about the user from other sources that the media guidance application may access. As a result, a user can be provided with a unified guidance application experience across the user's different user equipment devices. This type of user experience is described in greater detail below in connection with FIG. 4. Additional personalized media guidance application features are described in greater detail in Ellis et al., U.S. Patent Application Publication No. 2005/0251827, filed Jul. 11, 2005, Boyer et al., U.S. Pat. No. 7,165,098, issued Jan. 16, 2007, and Ellis et al., U.S. Patent Application Publication No. 2002/0174430, filed Feb. 21, 2002, which are hereby incorporated by reference herein in their entireties.

Another display arrangement for providing media guidance is shown in FIG. 2. Video mosaic display 200 includes selectable options 202 for content information organized based on content type, genre, and/or other organization criteria. In display 200, window 206 provides options for the enhanced nap feature implemented via the methods and systems described herein. The viewer may activate window 206 by selecting option 218 relating to the enhanced nap feature. The viewer may select option 220 to activate the learning assist feature. For example, the learning assist feature may initiate playback of a foreign language audio lesson when the user is detected to be falling asleep. The viewer may select option 222 for soothing sounds. For example, the soothing sounds feature may initiate playback of soothing ocean waves to help the user fall into deeper sleep. The viewer may select option 224 to turn off the enhanced nap feature.

In display 200, television listings option 204 is selected, thus providing listings 208, 210, and 212 as broadcast program listings. In display 200, the listings may provide graphical images including cover art, still images from the content, video clip previews, live video from the content, or other types of content that indicate to a user the content being described by the media guidance data in the listing. Each of the graphical listings may also be accompanied by text to provide further information about the content associated with the listing. For example, listing 208 may include more than one portion, including media portion 214 and text portion 216. Media portion 214 and/or text portion 216 may be selectable to view content in full-screen or to view information related to the content displayed in media portion 214 (for example, to view listings for the channel that the video is displayed on).

The listings in display 200 may be of different sizes, but if desired, all the listings may be the same size. Listings may be of different sizes or graphically accentuated to indicate degrees of interest to the user or to emphasize certain content, as desired by the content provider or based on user preferences. Various systems and methods for graphically accentuating content listings are discussed in, for example, Yates, U.S. Patent Application Publication No. 2010/0153885, filed Nov. 12, 2009, which is hereby incorporated by reference herein in its entirety.

Users may access content and the media guidance application (and its display screens described above and below) from one or more of their user equipment devices. FIG. 3 shows a generalized embodiment of illustrative user equipment device 300. More specific implementations of user equipment devices are discussed below in connection with FIG. 4. User equipment device 300 may receive content and data via input/output (hereinafter “I/O”) path 302. I/O path 302 may provide content (for example, broadcast programming, on-demand programming, Internet content, content available over a local area network (LAN) or wide area network (WAN), and/or other content) and data to control circuitry 304, which includes processing circuitry 306 and storage 308. Control circuitry 304 may be used to send and receive commands, requests, and other suitable data using I/O path 302. I/O path 302 may connect control circuitry 304 (and specifically processing circuitry 306) to one or more communications paths (described below). I/O functions may be provided by one or more of these communications paths, but are shown as a single path in FIG. 3 to avoid overcomplicating the drawing.

Control circuitry 304 may be based on any suitable processing circuitry such as processing circuitry 306. As referred to herein, processing circuitry should be understood to mean circuitry based on one or more microprocessors, microcontrollers, digital signal processors, programmable logic devices, field-programmable gate arrays (FPGAs), application-specific integrated circuits (ASICs), etc., and may include a multi-core processor (for example, dual-core, quad-core, hexa-core, or any suitable number of cores) or supercomputer. In some embodiments, processing circuitry may be distributed across multiple separate processors or processing units, for example, multiple of the same type of processing units (for example, two Intel Core i7 processors) or multiple different processors (for example, an Intel Core i5 processor and an Intel Core i7 processor). In some embodiments, control circuitry 304 executes instructions for a media guidance application stored in memory (that is, storage 308). Specifically, control circuitry 304 may be instructed by the media guidance application to perform the functions discussed above and below. For example, the media guidance application may provide instructions to control circuitry 304 to generate the media guidance displays. In some implementations, any action performed by control circuitry 304 may be based on instructions received from the media guidance application.

In client-server based embodiments, control circuitry 304 may include communications circuitry suitable for communicating with a guidance application server or other networks or servers. The instructions for carrying out the above mentioned functionality may be stored on the guidance application server. Communications circuitry may include a cable modem, an integrated services digital network (ISDN) modem, a digital subscriber line (DSL) modem, a telephone modem, Ethernet card, or a wireless modem for communications with other equipment, or any other suitable communications circuitry. Such communications may involve the Internet or any other suitable communications networks or paths (which is described in more detail in connection with FIG. 4). In addition, communications circuitry may include circuitry that enables peer-to-peer communication of user equipment devices, or communication of user equipment devices in locations remote from each other (described in more detail below).

Memory may be an electronic storage device provided as storage 308 that is part of control circuitry 304. As referred to herein, the phrase “electronic storage device” or “storage device” should be understood to mean any device for storing electronic data, computer software, or firmware, such as random-access memory, read-only memory, hard drives, optical drives, digital video disc (DVD) recorders, compact disc (CD) recorders, BLU-RAY disc (BD) recorders, BLU-RAY three dimensional disc recorders, digital video recorders (DVR, sometimes called a personal video recorder, or PVR), solid state devices, quantum storage devices, gaming consoles, gaming media, or any other suitable fixed or removable storage devices, and/or any combination of the same. Storage 308 may be used to store various types of content described herein as well as media guidance data described above. Nonvolatile memory may also be used (for example, to launch a boot-up routine and other instructions). Cloud-based storage, described in relation to FIG. 4, may be used to supplement storage 308 or instead of storage 308.

Control circuitry 304 may include video generating circuitry and tuning circuitry, such as one or more analog tuners, one or more MPEG-2 decoders or other digital decoding circuitry, high-definition tuners, or any other suitable tuning or video circuits or combinations of such circuits. Encoding circuitry (for example, for converting over-the-air, analog, or digital signals to MPEG signals for storage) may also be provided. Control circuitry 304 may also include scaler circuitry for upconverting and downconverting content into the preferred output format of the user equipment 300. Circuitry 304 may also include digital-to-analog converter circuitry and analog-to-digital converter circuitry for converting between digital and analog signals. The tuning and encoding circuitry may be used by the user equipment device to receive and to display, to play, or to record content. The tuning and encoding circuitry may also be used to receive guidance data. The circuitry described herein, including for example, the tuning, video generating, encoding, decoding, encrypting, decrypting, scaler, and analog/digital circuitry, may be implemented using software running on one or more general purpose or specialized processors. Multiple tuners may be provided to handle simultaneous tuning functions (for example, watch and record functions, picture-in-picture (PIP) functions, multiple-tuner recording, etc.). If storage 308 is provided as a separate device from user equipment 300, the tuning and encoding circuitry (including multiple tuners) may be associated with storage 308.

A user may send instructions to control circuitry 304 using user input interface 310. User input interface 310 may be any suitable user interface, such as a remote control, mouse, trackball, keypad, keyboard, touch screen, touchpad, stylus input, joystick, voice recognition interface, or other user input interfaces. Display 312 may be provided as a stand-alone device or integrated with other elements of user equipment device 300. For example, display 312 may be a touchscreen or touch-sensitive display. In such circumstances, user input interface 310 may be integrated with or combined with display 312. Display 312 may be one or more of a monitor, a television, a liquid crystal display (LCD) for a mobile device, amorphous silicon display, low temperature poly silicon display, electronic ink display, electrophoretic display, active matrix display, electro-wetting display, electrofluidic display, cathode ray tube display, light-emitting diode display, electroluminescent display, plasma display panel, high-performance addressing display, thin-film transistor display, organic light-emitting diode display, surface-conduction electron-emitter display (SED), laser television, carbon nanotubes, quantum dot display, interferometric modulator display, or any other suitable equipment for displaying visual images. In some embodiments, display 312 may be HDTV-capable. In some embodiments, display 312 may be a three dimensional display, and the interactive media guidance application and any suitable content may be displayed in three dimensional format. A video card or graphics card may generate the output to the display 312. The video card may offer various functions such as accelerated rendering of three dimensional scenes and two dimensional graphics, MPEG-2/MPEG-4 decoding, TV output, or the ability to connect multiple monitors. The video card may be any processing circuitry described above in relation to control circuitry 304. The video card may be integrated with the control circuitry 304. Speakers 314 may be provided as integrated with other elements of user equipment device 300 or may be stand-alone units. The audio component of videos and other content displayed on display 312 may be played through speakers 314. In some embodiments, the audio may be distributed to a receiver (not shown), which processes and outputs the audio via speakers 314.

Detection device 316 may be provided as integrated with other elements of user equipment device 300 or may be a stand-alone unit. The interactive media guidance application receives measurements from the detection device relating to the user's sleep state from the user. For example, the interactive media guidance application may receive a position of the user from a camera detection device (for example, sitting up indicating awake or lying down indicating asleep), a sound from or near the user from a microphone detection device (for example, cheering sounds indicating awake or light snoring indicating asleep), a heart rate from a biometric sensor detection device on the user (for example, a higher heart rate indicating awake or a lower heart rate indicating asleep), brain activity from an electroencephalographic (EEG) detection device, or another suitable measurement from the detection device relating to the user's sleep state. One or more of these measurements may be included in a data structure representing the user's sleep state.

As discussed above, the sleep state indicates the current state of the user, for example, whether the user is awake, whether the user is asleep, or another suitable state of the user. In some embodiments, the sleep state indicates one of several states of the user. For example, the sleep state may indicate whether the user is dreaming (for example, when the user is experiencing REM sleep), whether the user is in deep sleep (for example, when the user is experiencing slow-wave sleep), whether the user is alert dozing (for example, when the user is experiencing NREM sleep), whether the user is sleepy (for example, when the user's heart rate is below a certain threshold), whether the user is awake (for example, when the user's heart rate is above a certain threshold), or another suitable state of the user.

The guidance application may be implemented using any suitable architecture. For example, it may be a stand-alone application wholly-implemented on user equipment device 300. In such an approach, instructions of the application are stored locally (for example, in storage 308), and data for use by the application is downloaded on a periodic basis (for example, from an out-of-band feed, from an Internet resource, or using another suitable approach). Control circuitry 304 may retrieve instructions of the application from storage 308 and process the instructions to generate any of the displays discussed herein. Based on the processed instructions, control circuitry 304 may determine what action to perform when input is received from input interface 310. For example, movement of a cursor on a display up/down may be indicated by the processed instructions when input interface 310 indicates that an up/down button was selected.

In some embodiments, the media guidance application is a client-server based application. Data for use by a thick or thin client implemented on user equipment device 300 is retrieved on-demand by issuing requests to a server remote to the user equipment device 300. In one example of a client-server based guidance application, control circuitry 304 runs a web browser that interprets web pages provided by a remote server. For example, the remote server may store the instructions for the application in a storage device. The remote server may process the stored instructions using circuitry (for example, control circuitry 304) and generate the displays discussed above and below. The client device may receive the displays generated by the remote server and may display the content of the displays locally on equipment device 300. This way, the processing of the instructions is performed remotely by the server while the resulting displays are provided locally on equipment device 300. Equipment device 300 may receive inputs from the user via input interface 310 and transmit those inputs to the remote server for processing and generating the corresponding displays. For example, equipment device 300 may transmit a communication to the remote server indicating that an up/down button was selected via input interface 310. The remote server may process instructions in accordance with that input and generate a display of the application corresponding to the input (for example, a display that moves a cursor up/down). The generated display is then transmitted to equipment device 300 for presentation to the user.

In some embodiments, the media guidance application is downloaded and interpreted or otherwise run by an interpreter or virtual machine (run by control circuitry 304). In some embodiments, the guidance application may be encoded in the ETV Binary Interchange Format (EBIF), received by control circuitry 304 as part of a suitable feed, and interpreted by a user agent running on control circuitry 304. For example, the guidance application may be an EBIF application. In some embodiments, the guidance application may be defined by a series of JAVA-based files that are received and run by a local virtual machine or other suitable middleware executed by control circuitry 304. In some of such embodiments (for example, those employing MPEG-2 or other digital media encoding schemes), the guidance application may be, for example, encoded and transmitted in an MPEG-2 object carousel with the MPEG audio and video packets of a program.

User equipment device 300 of FIG. 3 can be implemented in system 400 of FIG. 4 as user television equipment 402, user computer equipment 404, wireless user communications device 406, or any other type of user equipment suitable for accessing content, such as a non-portable gaming machine. For simplicity, these devices may be referred to herein collectively as user equipment or user equipment devices, and may be substantially similar to user equipment devices described above. User equipment devices, on which a media guidance application may be implemented, may function as a standalone device or may be part of a network of devices. Various network configurations of devices may be implemented and are discussed in more detail below.

A user equipment device utilizing at least some of the system features described above in connection with FIG. 3 may not be classified solely as user television equipment 402, user computer equipment 404, or a wireless user communications device 406. For example, user television equipment 402 may, like some user computer equipment 404, be Internet-enabled allowing for access to Internet content, while user computer equipment 404 may, like some television equipment 402, include a tuner allowing for access to television programming. The media guidance application may have the same layout on various different types of user equipment or may be tailored to the display capabilities of the user equipment. For example, on user computer equipment 404, the guidance application may be provided as a web site accessed by a web browser. In another example, the guidance application may be scaled down for wireless user communications devices 406.

In system 400, there is typically more than one of each type of user equipment device but only one of each is shown in FIG. 4 to avoid overcomplicating the drawing. In addition, each user may utilize more than one type of user equipment device and also more than one of each type of user equipment device.

In some embodiments, a user equipment device (for example, user television equipment 402, user computer equipment 404, wireless user communications device 406) may be referred to as a “second screen device.” For example, a second screen device may supplement content presented on a first user equipment device. The content presented on the second screen device may be any suitable content that supplements the content presented on the first device. In some embodiments, the second screen device provides an interface for adjusting settings and display preferences of the first device. In some embodiments, the second screen device is configured for interacting with other second screen devices or for interacting with a social network. The second screen device can be located in the same room as the first device, a different room from the first device but in the same house or building, or in a different building from the first device.

The user may also set various settings to maintain consistent media guidance application settings across in-home devices and remote devices. Settings include those described herein, as well as channel and program favorites, programming preferences that the guidance application utilizes to make programming recommendations, display preferences, and other desirable guidance settings. For example, if a user sets a channel as a favorite on, for example, the web site www.allrovi.com on their personal computer at their office, the same channel would appear as a favorite on the user's in-home devices (for example, user television equipment and user computer equipment) as well as the user's mobile devices, if desired. Therefore, changes made on one user equipment device can change the guidance experience on another user equipment device, regardless of whether they are the same or a different type of user equipment device. In addition, the changes made may be based on settings input by a user, as well as user activity monitored by the guidance application.

The user equipment devices may be coupled to communications network 414. Namely, user television equipment 402, user computer equipment 404, and wireless user communications device 406 are coupled to communications network 414 via communications paths 408, 410, and 412, respectively. Communications network 414 may be one or more networks including the Internet, a mobile phone network, mobile voice or data network (for example, a 4G or LTE network), cable network, public switched telephone network, or other types of communications network or combinations of communications networks. Paths 408, 410, and 412 may separately or together include one or more communications paths, such as, a satellite path, a fiber-optic path, a cable path, a path that supports Internet communications (for example, IPTV), free-space connections (for example, for broadcast or other wireless signals), or any other suitable wired or wireless communications path or combination of such paths. Path 412 is drawn with dotted lines to indicate that in the exemplary embodiment shown in FIG. 4 it is a wireless path and paths 408 and 410 are drawn as solid lines to indicate they are wired paths (although these paths may be wireless paths, if desired). Communications with the user equipment devices may be provided by one or more of these communications paths, but are shown as a single path in FIG. 4 to avoid overcomplicating the drawing.

Although communications paths are not drawn between user equipment devices; these devices may communicate directly with each other via communication paths, such as those described above in connection with paths 408, 410, and 412, as well as other short-range point-to-point communication paths, such as USB cables, IEEE 1394 cables, wireless paths (for example, Bluetooth, infrared, IEEE 802-11x, etc.), or other short-range communication via wired or wireless paths. BLUETOOTH is a certification mark owned by Bluetooth SIG, INC. The user equipment devices may also communicate with each other directly through an indirect path via communications network 414.

System 400 includes content source 416, media guidance data source 418, and reference sleep state source 424 coupled to communications network 414 via communication paths 420, 422, and 426, respectively. Paths 420 and 422 may include any of the communication paths described above in connection with paths 408, 410, and 412. Communications with the content source 416 and media guidance data source 418 may be exchanged over one or more communications paths, but are shown as a single path in FIG. 4 to avoid overcomplicating the drawing. In addition, there may be more than one of each of content source 416 and media guidance data source 418, but only one of each is shown in FIG. 4 to avoid overcomplicating the drawing. The different types of each of these sources are discussed below. If desired, content source 416 and media guidance data source 418 may be integrated as one source device. Although communications between sources 416 and 418 with user equipment devices 402, 404, and 406 are shown as through communications network 414, in some embodiments, sources 416 and 418 may communicate directly with user equipment devices 402, 404, and 406 via communication paths (not shown) such as those described above in connection with paths 408, 410, and 412.

Content source 416 may include one or more types of content distribution equipment including a television distribution facility, cable system headend, satellite distribution facility, programming sources (for example, television broadcasters, such as NBC, ABC, HBO, etc.), intermediate distribution facilities and/or servers, Internet providers, on-demand media servers, and other content providers. NBC is a trademark owned by the National Broadcasting Company, Inc., ABC is a trademark owned by the American Broadcasting Company, Inc., and HBO is a trademark owned by the Home Box Office, Inc. Content source 416 may be the originator of content (for example, a television broadcaster, a Webcast provider, etc.) or may not be the originator of content (for example, an on-demand content provider, an Internet provider of content of broadcast programs for downloading, etc.). Content source 416 may include cable sources, satellite providers, on-demand providers, Internet providers, over-the-top content providers, or other providers of content. Content source 416 may also include a remote media server used to store different types of content (including video content selected by a user), in a location remote from any of the user equipment devices. Systems and methods for remote storage of content, and providing remotely stored content to user equipment are discussed in greater detail in connection with Ellis et al., U.S. Pat. No. 7,761,892, issued Jul. 20, 2010, which is hereby incorporated by reference herein in its entirety.

Media guidance data source 418 may provide media guidance data, such as the media guidance data described above. Media guidance data may be provided to the user equipment devices using any suitable approach. In some embodiments, the guidance application may be a stand-alone interactive television program guide that receives program guide data via a data feed (for example, a continuous feed or trickle feed). Program schedule data and other guidance data may be provided to the user equipment on a television channel sideband, using an in-band digital signal, using an out-of-band digital signal, or by any other suitable data transmission technique. Program schedule data and other media guidance data may be provided to user equipment on multiple analog or digital television channels.

Reference sleep state source 424 may implement part or all of the systems and methods described herein for enhancing sleep of a user of an interactive media guidance application. Reference sleep state source 424 may retrieve and/or provide information regarding reference sleep states relating to a user that is about to fall asleep or a user that is ready to be awakened. The reference sleep states may pertain to a reference user or the previously measured sleep states for the current user. Reference sleep state source 424 may assist in enhancing sleep of the user by determining the user's current sleep state and one or more media assets related to the user's current sleep state. Reference sleep state source 424 may be a television distribution server, cable system headend, satellite distribution server, programming sources server, intermediate distribution facilities and/or servers, an Internet provider server, an on-demand media server, or another suitable server.

In some embodiments, guidance data from media guidance data source 418 may be provided to users' equipment using a client-server approach. For example, a user equipment device may pull media guidance data from a server, or a server may push media guidance data to a user equipment device. In some embodiments, a guidance application client residing on the user's equipment may initiate sessions with source 418 to obtain guidance data when needed, for example, when the guidance data is out of date or when the user equipment device receives a request from the user to receive data. Media guidance may be provided to the user equipment with any suitable frequency (for example, continuously, daily, a user-specified period of time, a system-specified period of time, in response to a request from user equipment, etc.). Media guidance data source 418 may provide user equipment devices 402, 404, and 406 the media guidance application itself or software updates for the media guidance application.

In some embodiments, the media guidance data may include viewer data. For example, the viewer data may include current and/or historical user activity information (for example, what content the user typically watches, what times of day the user watches content, whether the user interacts with a social network, at what times the user interacts with a social network to post information, what types of content the user typically watches (for example, pay TV or free TV), mood, brain activity information, etc.). The media guidance data may also include subscription data. For example, the subscription data may identify to which sources or services a given user subscribes and/or to which sources or services the given user has previously subscribed but later terminated access (for example, whether the user subscribes to premium channels, whether the user has added a premium level of services, whether the user has increased Internet speed). In some embodiments, the viewer data and/or the subscription data may identify patterns of a given user for a period of more than one year. The media guidance data may include a model (for example, a survivor model) used for generating a score that indicates a likelihood a given user will terminate access to a service/source. For example, the media guidance application may process the viewer data with the subscription data using the model to generate a value or score that indicates a likelihood of whether the given user will terminate access to a particular service or source. In particular, a higher score may indicate a higher level of confidence that the user will terminate access to a particular service or source. Based on the score, the media guidance application may generate promotions and advertisements that entice the user to keep the particular service or source indicated by the score as one to which the user will likely terminate access.

Media guidance applications may be, for example, stand-alone applications implemented on user equipment devices. For example, the media guidance application may be implemented as software or a set of executable instructions which may be stored in storage 308, and executed by control circuitry 304 of a user equipment device 300. In some embodiments, media guidance applications may be client-server applications where only a client application resides on the user equipment device, and server application resides on a remote server. For example, media guidance applications may be implemented partially as a client application on control circuitry 304 of user equipment device 300 and partially on a remote server as a server application (for example, media guidance data source 418) running on control circuitry of the remote server. When executed by control circuitry of the remote server (such as media guidance data source 418), the media guidance application may instruct the control circuitry to generate the guidance application displays and transmit the generated displays to the user equipment devices. The server application may instruct the control circuitry of the media guidance data source 418 to transmit data for storage on the user equipment. The client application may instruct control circuitry of the receiving user equipment to generate the guidance application displays.

Content and/or media guidance data delivered to user equipment devices 402, 404, and 406 may be over-the-top (OTT) content. OTT content delivery allows Internet-enabled user devices, including any user equipment device described above, to receive content that is transferred over the Internet, including any content described above, in addition to content received over cable or satellite connections. OTT content is delivered via an Internet connection provided by an Internet service provider (ISP), but a third party distributes the content. The ISP may not be responsible for the viewing abilities, copyrights, or redistribution of the content, and may only transfer IP packets provided by the OTT content provider. Examples of OTT content providers include YOUTUBE, NETFLIX, and HULU, which provide audio and video via IP packets. Youtube is a trademark owned by Google Inc., Netflix is a trademark owned by Netflix Inc., and Hulu is a trademark owned by Hulu, LLC. OTT content providers may additionally or alternatively provide media guidance data described above. In addition to content and/or media guidance data, providers of OTT content can distribute media guidance applications (for example, web-based applications or cloud-based applications), or the content can be displayed by media guidance applications stored on the user equipment device.

Media guidance system 400 is intended to illustrate a number of approaches, or network configurations, by which user equipment devices and sources of content and guidance data may communicate with each other for the purpose of accessing content and providing media guidance. The embodiments described herein may be applied in any one or a subset of these approaches, or in a system employing other approaches for delivering content and providing media guidance. The following four approaches provide specific illustrations of the generalized example of FIG. 4.

In one approach, user equipment devices may communicate with each other within a home network. User equipment devices can communicate with each other directly via short-range point-to-point communication schemes described above, via indirect paths through a hub or other similar device provided on a home network, or via communications network 414. Each of the multiple individuals in a single home may operate different user equipment devices on the home network. As a result, it may be desirable for various media guidance information or settings to be communicated between the different user equipment devices. For example, it may be desirable for users to maintain consistent media guidance application settings on different user equipment devices within a home network, as described in greater detail in Ellis et al., U.S. Patent Publication No. 2005/0251827, filed Jul. 11, 2005. Different types of user equipment devices in a home network may also communicate with each other to transmit content. For example, a user may transmit content from user computer equipment to a portable video player or portable music player.

In a second approach, users may have multiple types of user equipment by which they access content and obtain media guidance. For example, some users may have home networks that are accessed by in-home and mobile devices. Users may control in-home devices via a media guidance application implemented on a remote device. For example, users may access an online media guidance application on a website via a personal computer at their office, or a mobile device such as a PDA or web-enabled mobile telephone. The user may set various settings (for example, recordings, reminders, or other settings) on the online guidance application to control the user's in-home equipment. The online guide may control the user's equipment directly, or by communicating with a media guidance application on the user's in-home equipment. Various systems and methods for user equipment devices communicating, where the user equipment devices are in locations remote from each other, is discussed in, for example, Ellis et al., U.S. Pat. No. 8,046,801, issued Oct. 25, 2011, which is hereby incorporated by reference herein in its entirety.

In a third approach, users of user equipment devices inside and outside a home can use their media guidance application to communicate directly with content source 416 to access content. Specifically, within a home, users of user television equipment 402 and user computer equipment 404 may access the media guidance application to navigate among and locate desirable content. Users may also access the media guidance application outside of the home using wireless user communications devices 406 to navigate among and locate desirable content.

In a fourth approach, user equipment devices may operate in a cloud computing environment to access cloud services. In a cloud computing environment, various types of computing services for content sharing, storage or distribution (for example, video sharing sites or social networking sites) are provided by a collection of network-accessible computing and storage resources, referred to as “the cloud.” For example, the cloud can include a collection of server computing devices, which may be located centrally or at distributed locations, that provide cloud-based services to various types of users and devices connected via a network such as the Internet via communications network 414. These cloud resources may include one or more content sources 416 and one or more media guidance data sources 418. In addition or in the alternative, the remote computing sites may include other user equipment devices, such as user television equipment 402, user computer equipment 404, and wireless user communications device 406. For example, the other user equipment devices may provide access to a stored copy of a video or a streamed video. In such embodiments, user equipment devices may operate in a peer-to-peer manner without communicating with a central server.

The cloud provides access to services, such as content storage, content sharing, or social networking services, among other examples, as well as access to any content described above, for user equipment devices. Services can be provided in the cloud through cloud computing service providers, or through other providers of online services. For example, the cloud-based services can include a content storage service, a content sharing site, a social networking site, or other services via which user-sourced content is distributed for viewing by others on connected devices. These cloud-based services may allow a user equipment device to store content to the cloud and to receive content from the cloud rather than storing content locally and accessing locally-stored content.

A user may use various content capture devices, such as camcorders, digital cameras with video mode, audio recorders, mobile phones, and handheld computing devices, to record content. The user can upload content to a content storage service on the cloud either directly, for example, from user computer equipment 404 or wireless user communications device 406 having content capture feature. Alternatively, the user can first transfer the content to a user equipment device, such as user computer equipment 404. The user equipment device storing the content uploads the content to the cloud using a data transmission service on communications network 414. In some embodiments, the user equipment device itself is a cloud resource, and other user equipment devices can access the content directly from the user equipment device on which the user stored the content.

Cloud resources may be accessed by a user equipment device using, for example, a web browser, a media guidance application, a desktop application, a mobile application, and/or any combination of access applications of the same. The user equipment device may be a cloud client that relies on cloud computing for application delivery, or the user equipment device may have some functionality without access to cloud resources. For example, some applications running on the user equipment device may be cloud applications, that is, applications delivered as a service over the Internet, while other applications may be stored and run on the user equipment device. In some embodiments, a user device may receive content from multiple cloud resources simultaneously. For example, a user device can stream audio from one cloud resource while downloading content from a second cloud resource. Or a user device can download content from multiple cloud resources for more efficient downloading. In some embodiments, user equipment devices can use cloud resources for processing operations such as the processing operations performed by processing circuitry described in relation to FIG. 3.

As referred herein, the term “in response to” refers to initiated as a result of. For example, a first action being performed in response to another action may include interstitial steps between the first action and the second action. As referred herein, the term “directly in response to” refers to caused by. For example, a first action being performed directly in response to another action may not include interstitial steps between the first action and the second action.

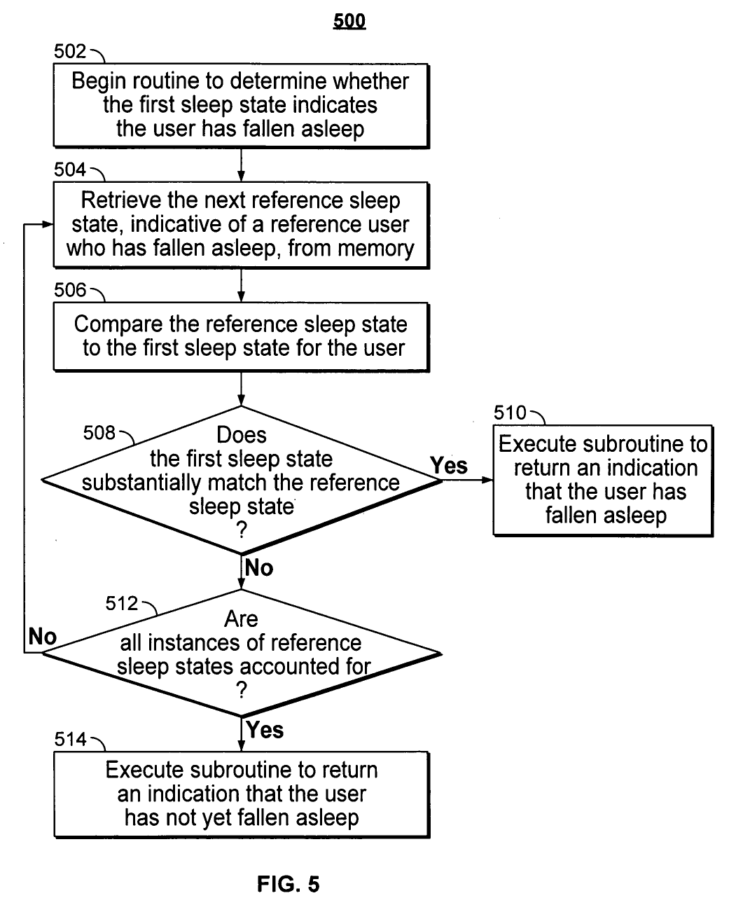

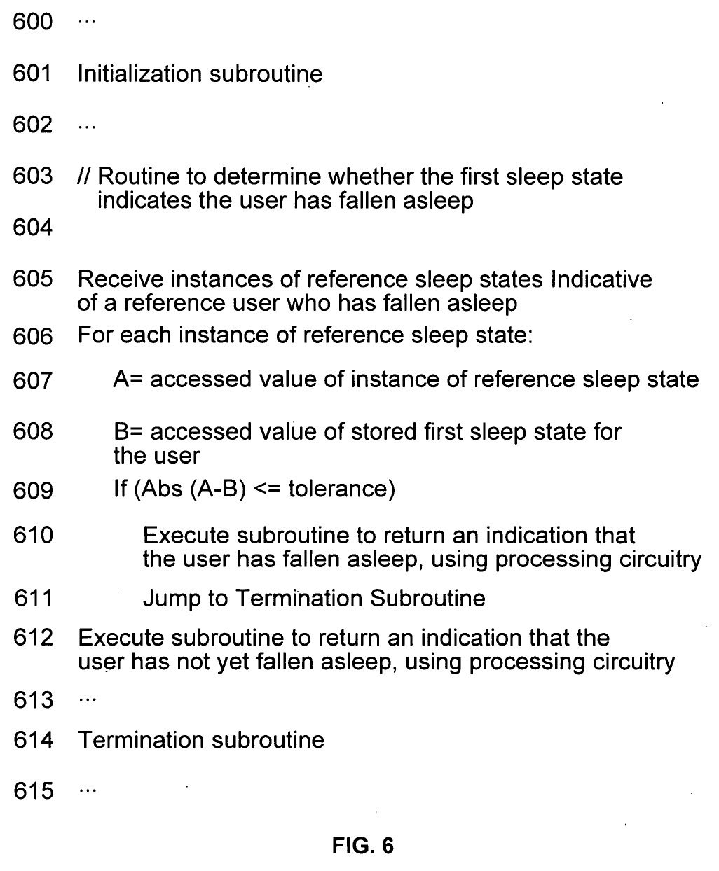

FIGS. 5 and 6 present an algorithm for control circuitry (for example, control circuitry 304) to determine whether the first sleep state indicates the user has fallen asleep in accordance with some embodiments of the disclosure. In some embodiments, this algorithm may be encoded on to non-transitory storage medium (for example, storage device 308) as a set of instructions to be decoded and executed by processing circuitry (for example, processing circuitry 306). Processing circuitry may in turn provide instructions to other sub-circuits contained within control circuitry 304, such as the tuning, video generating, encoding, decoding, encrypting, decrypting, scaling, analog/digital conversion circuitry, and the like.

The flowchart in FIG. 5 describes a process implemented on control circuitry (for example, control circuitry 304) to determine whether the first sleep state indicates the user has fallen asleep in accordance with some embodiments of the disclosure. At step 502, control circuitry 304 begins a routine to determine whether the first sleep state indicates the user has fallen asleep. In some embodiments, this may be done either directly or indirectly in response to a user action or input (for example, from signals received by control circuitry 304 or user input interface 310). For example, the process may begin directly in response to control circuitry 304 receiving signals from user input interface 310 or detection device 316, or control circuitry 304 may prompt the user to confirm his or her input using a display (for example, by generating a prompt to be displayed on display 312) prior to running the algorithm.

At step 504, control circuitry 304 proceeds to retrieve the next reference sleep state, indicative of a reference user who has fallen asleep, from memory (for example, storage device 308). In some embodiments, control circuitry 304 may receive a single primitive data structure that represents the reference sleep state. At step 506, control circuitry 304 proceeds to compare the reference sleep state to the first sleep state for the user. In some embodiments, the reference sleep state may be stored as part of a larger data structure, and control circuitry 304 may retrieve the information relating to the reference sleep state by executing appropriate accessor methods on the larger data structure.

At step 508, control circuitry 304 proceeds to determine whether the first sleep state substantially matches the reference sleep state. In some embodiments, the first sleep state may be stored (for example, on storage device 308) prior to beginning the process. In some embodiments, control circuitry 304 may directly compare the values of the first sleep state and the reference sleep state by accessing the values respectively from memory and performing a value comparison. In some instances, control circuitry 304 may call a comparison function (for example, for object to object comparison) to compare the first sleep state and the reference sleep state. If the condition is satisfied, the algorithm may proceed to step 510; if the condition is not satisfied, the algorithm may proceed to step 512 instead.

At step 510, control circuitry 304 executes a subroutine to return an indication that the user has fallen asleep based on the condition at step 508 being satisfied. After the subroutine is executed, the algorithm may proceed to terminate the process.

At step 512, control circuitry 304 checks if all instances of reference sleep states are accounted for. If all of the instances have been evaluated, control circuitry 304 may proceed to step 514. For example, control circuitry 304 may call a function to see if there is a next instance of the reference sleep state. If the function returns true (that is, there are still instances that need to be processed), control circuitry 304 may proceed to step 504.

At step 514, control circuitry 304 executes a subroutine to return an indication that the user has not yet fallen asleep. Because the first sleep state did not match any of the reference sleep states indicative of a reference user who has fallen asleep, the user has likely not yet fallen asleep.

It is contemplated that the descriptions of FIG. 5 may be used with any other embodiment of this disclosure. In addition, the descriptions described in relation to the algorithm of FIG. 5 may be done in alternative orders or in parallel to further the purposes of this disclosure. For example, conditional statements and logical evaluations may be performed in any order or in parallel or simultaneously to reduce lag or increase the speed of the system or method. As a further example, in some embodiments, several instances of reference sleep states may be evaluated in parallel, using multiple logical processor threads or the algorithm may be enhanced by incorporating branch prediction. Furthermore, it should be noted that the process of FIG. 5 may be implemented on a combination of appropriately configured software and hardware, and that any of the devices or equipment discussed in relation to FIGS. 3-4 could be used to implement one or more portions of the process.

The pseudocode in FIG. 6 describes a process to determine whether the first sleep state indicates the user has fallen asleep in accordance with some embodiments of the disclosure. It will be evident to one skilled in the art that the process described by the pseudocode in FIG. 6 may be implemented in any number of programming languages and a variety of different hardware, and that the style and format should not be construed as limiting, but rather a general template of the steps and procedures that would be consistent with code used to implement some embodiments of this disclosure.

At line 601, control circuitry 304 runs a subroutine to initialize variables and prepare to determine whether the first sleep state indicates the user has fallen asleep, which begins on line 605. For example, in some embodiments, control circuitry 304 may copy instructions from non-transitory storage medium (for example, storage device 308) into RAM or into the cache for processing circuitry 306 during the initialization stage. Additionally, in some embodiments, additional information relating to a subroutine may be retrieved, set, and stored at 601.

At line 605, control circuitry 304 retrieves instances of reference sleep states, indicative of a reference user who has fallen asleep, from memory (for example, storage device 308). Control circuitry 304 may retrieve the instances of reference sleep states by receiving, for example, a pointer to an array for multiple reference sleep states. In another example, control circuitry 304 may receive an object of a class, such as an iterator object containing elements of instances of reference sleep states.

At line 606, control circuitry 304 iterates through the instances of reference sleep states indicative of a reference user who has fallen asleep. If only a single reference sleep state is indicated, the loop will only execute once. This loop may be implemented in multiple fashions depending on the choice of hardware and software language used to implement the algorithm of FIG. 6. For example, this may be implemented as part of a “for” or “while” loop.

At line 607, control circuitry 304 stores the data structure for the reference sleep state into a temporary variable “A.” In some embodiments, the reference sleep state will be stored as part of a larger data structure or class, and the reference sleep state may be obtained through appropriate accessor methods. In some embodiments, the reference sleep state may be converted from a string or other non-numeric data type into a numeric data type by means of an appropriate hashing algorithm. In some embodiments, control circuitry 304 may call a function to perform a comparison of the reference sleep state to the first sleep state. In some embodiments, the reference sleep state may be encoded as a primitive data structure, and rather than using a temporary variable, the reference sleep state may be directly used in the comparison at line 609.

At line 608, control circuitry 304 stores the first sleep state into a temporary variable “B.” Similar to the reference sleep state, in some embodiments, the first sleep state will be stored as part of a larger data structure or class, and the first sleep state may be obtained through accessor methods. In some embodiments, the first sleep state may be converted from a string or other non-numeric data type into a numeric data type by means of an appropriate hashing algorithm, or the first sleep state may be a primitive data structure, and may be directly used in the comparison at lines 609.

At line 609, control circuitry 304 compares the value of A to the value of B to see if they are essentially equivalent. This is achieved by subtracting the value of B from A, taking the absolute value of the difference, and then comparing the absolute value of the difference to a predetermined tolerance level. In some embodiments, the tolerance level may be a set percentage of either A or B. In some embodiments, the tolerance level may be a fixed number. For example, setting the tolerance level to a set multiple of machine epsilon may allow for the algorithm to account for small rounding errors that may result from the use of floating point arithmetic. In some embodiments, the tolerance level may be set to zero, or the condition inside the IF statement may be replaced with a strict equivalence between A and B.

At line 610, control circuitry 304 executes a subroutine to return an indication that the user has fallen asleep, using processing circuitry, if the condition in line 609 is satisfied. In some embodiments, this may be achieved by processing circuitry 306 sending the appropriate signals to control circuitry 304. At line 611, control circuitry 304 jumps to the termination subroutine and prepares to end the process.

At line 612, control circuitry 304 executes a subroutine to indicate that the user has not yet fallen asleep, using processing circuitry. Because the first sleep state did not match any of the reference sleep states indicative of a reference user who has fallen asleep, the user has likely not yet fallen asleep.

At line 614, control circuitry 304 runs a termination subroutine after the algorithm has performed its function. For example, in some embodiments, control circuitry 304 may destruct variables, perform garbage collection, free memory or clear the cache of processing circuitry 306.

It will be evident to one skilled in the art that the process described by the pseudocode in FIG. 6 may be implemented in any number of programming languages and a variety of different hardware, and the particular choice and location of primitive functions, logical evaluations, and function evaluations are not intended to be limiting. It will also be evident that the code may be refactored or rewritten to manipulate the order of the various logical evaluations, perform several iterations in parallel rather than in a single iterative loop, or to otherwise manipulate and optimize run-time and performance metrics without fundamentally changing the inputs or final outputs. For example, in some embodiments break conditions may be placed after lines 610 to speed operation, or the conditional statements may be replaced with a case-switch. In some embodiments, rather than iterating over all instances of reference sleep states at step 606, in some embodiments, the code may be rewritten so control circuitry 304 is instructed to evaluate multiple instances of reference sleep states simultaneously on a plurality of processors or processor threads, lowering the number of iterations needed and potentially speeding up computation time.

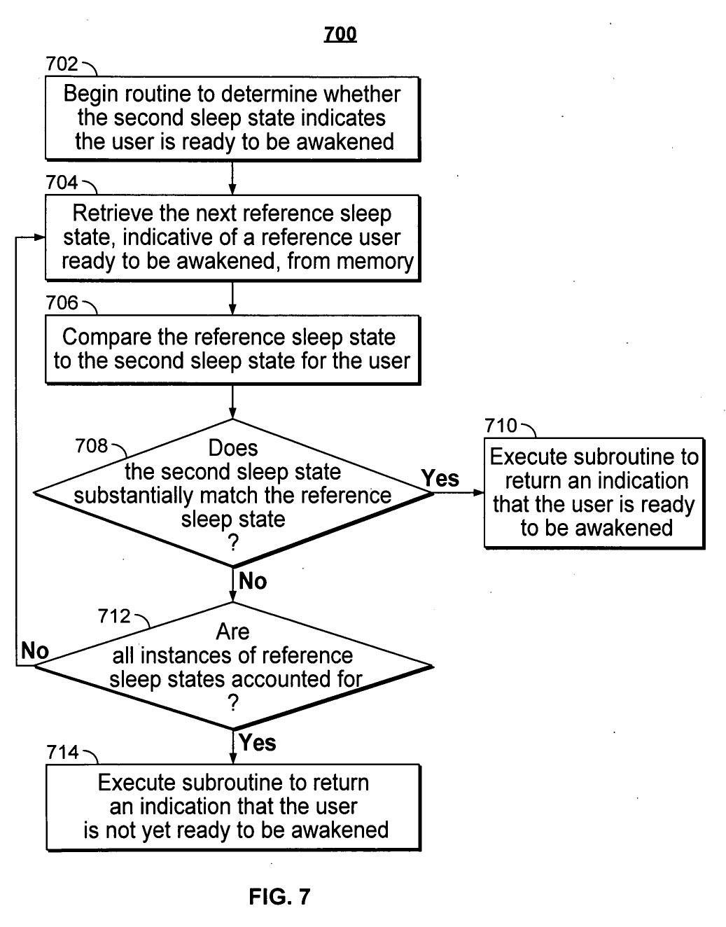

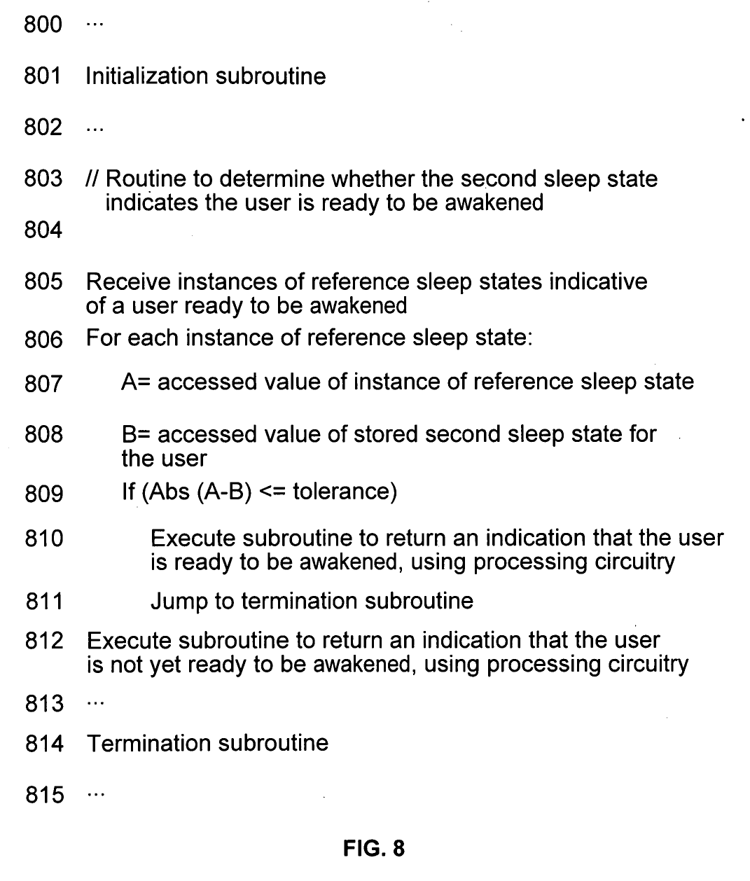

FIGS. 7 and 8 present an algorithm for control circuitry (for example, control circuitry 304) to determine whether the second sleep state indicates the user is ready to be awakened in accordance with some embodiments of the disclosure. In some embodiments, this algorithm may be encoded on to non-transitory storage medium (for example, storage device 308) as a set of instructions to be decoded and executed by processing circuitry (for example, processing circuitry 306). Processing circuitry may in turn provide instructions to other sub-circuits contained within control circuitry 304, such as the tuning, video generating, encoding, decoding, encrypting, decrypting, scaling, analog/digital conversion circuitry, and the like.

The flowchart in FIG. 7 describes a process implemented on control circuitry (for example, control circuitry 304) to determine whether the second sleep state indicates the user is ready to be awakened in accordance with some embodiments of the disclosure. At step 702, control circuitry 304 begins a routine to determine whether the second sleep state indicates the user is ready to be awakened. In some embodiments, this may be done either directly or indirectly in response to a user action or input (for example, from signals received by control circuitry 304 or user input interface 310). For example, the process may begin directly in response to control circuitry 304 receiving signals from user input interface 310 or detection device 316, or control circuitry 304 may prompt the user to confirm his or her input using a display (for example, by generating a prompt to be displayed on display 312) prior to running the algorithm.

At step 704, control circuitry 304 proceeds to retrieve the next reference sleep state, indicative of a reference user ready to be awakened, from memory (for example, storage device 308). In some embodiments, control circuitry 304 may receive a single primitive data structure that represents the reference sleep state. At step 706, control circuitry 304 proceeds to compare the reference sleep state to the second sleep state for the user. In some embodiments, the reference sleep state may be stored as part of a larger data structure, and control circuitry 304 may retrieve the information relating to the reference sleep state by executing appropriate accessor methods on the larger data structure.

At step 708, control circuitry 304 proceeds to determine whether the second sleep state substantially matches the reference sleep state. In some embodiments, the second sleep state may be stored (for example, on storage device 308) prior to beginning the process. In some embodiments, control circuitry 304 may directly compare the values of the second sleep state and the reference sleep state by accessing the values respectively from memory and performing a value comparison. In some instances, control circuitry 304 may call a comparison function (for example, for object to object comparison) to compare the second sleep state and the reference sleep state. If the condition is satisfied, the algorithm may proceed to step 710; if the condition is not satisfied, the algorithm may proceed to step 712 instead.

At step 710, control circuitry 304 executes a subroutine to return an indication that the user is ready to be awakened based on the condition at step 708 being satisfied. After the subroutine is executed, the algorithm may proceed to terminate the process.

At step 712, control circuitry 304 checks if all instances of reference sleep states are accounted for. If all of the instances have been evaluated, control circuitry 304 may proceed to step 714. For example, control circuitry 304 may call a function to see if there is a next instance of the reference sleep state. If the function returns true (that is, there are still instances that need to be processed), control circuitry 304 may proceed to step 704.

At step 714, control circuitry 304 executes a subroutine to return an indication that the user is not yet ready to be awakened. Because the second sleep state did not match any of the reference sleep states indicative of a reference user ready to be awakened, the user is likely not yet ready to be awakened.

It is contemplated that the descriptions of FIG. 7 may be used with any other embodiment of this disclosure. In addition, the descriptions described in relation to the algorithm of FIG. 7 may be done in alternative orders or in parallel to further the purposes of this disclosure. For example, conditional statements and logical evaluations may be performed in any order or in parallel or simultaneously to reduce lag or increase the speed of the system or method. As a further example, in some embodiments, several instances of reference sleep states may be evaluated in parallel, using multiple logical processor threads or the algorithm may be enhanced by incorporating branch prediction. Furthermore, it should be noted that the process of FIG. 7 may be implemented on a combination of appropriately configured software and hardware, and that any of the devices or equipment discussed in relation to FIGS. 3-4 could be used to implement one or more portions of the process.

The pseudocode in FIG. 8 describes a process to determine whether the second sleep state indicates the user is ready to be awakened in accordance with some embodiments of the disclosure. It will be evident to one skilled in the art that the process described by the pseudocode in FIG. 8 may be implemented in any number of programming languages and a variety of different hardware, and that the style and format should not be construed as limiting, but rather a general template of the steps and procedures that would be consistent with code used to implement some embodiments of this disclosure.

At line 801, control circuitry 304 runs a subroutine to initialize variables and prepare to determine whether the second sleep state indicates the user is ready to be awakened, which begins on line 805. For example, in some embodiments, control circuitry 304 may copy instructions from non-transitory storage medium (for example, storage device 308) into RAM or into the cache for processing circuitry 306 during the initialization stage. Additionally, in some embodiments, additional information relating to a subroutine may be retrieved, set, and stored at 801.

At line 805, control circuitry 304 retrieves instances of reference sleep states, indicative of a reference user ready to be awakened, from memory (for example, storage device 308). Control circuitry 304 may retrieve the instances of reference sleep states by receiving, for example, a pointer to an array for multiple reference sleep states. In another example, control circuitry 304 may receive an object of a class, such as an iterator object containing elements of instances of reference sleep states.

At line 806, control circuitry 304 iterates through the instances of reference sleep states indicative of a reference user ready to be awakened. If only a single reference sleep state is indicated, the loop will only execute once. This loop may be implemented in multiple fashions depending on the choice of hardware and software language used to implement the algorithm of FIG. 8. For example, this may be implemented as part of a “for” or “while” loop.

At line 807, control circuitry 304 stores the data structure for the reference sleep state into a temporary variable “A.” In some embodiments, the reference sleep state will be stored as part of a larger data structure or class, and the reference sleep state may be obtained through appropriate accessor methods. In some embodiments, the reference sleep state may be converted from a string or other non-numeric data type into a numeric data type by means of an appropriate hashing algorithm. In some embodiments, control circuitry 304 may call a function to perform a comparison of the reference sleep state to the second sleep state. In some embodiments, the reference sleep state may be encoded as a primitive data structure, and rather than using a temporary variable, the reference sleep state may be directly used in the comparison at line 609.

At line 808, control circuitry 304 stores the second sleep state into a temporary variable “B.” Similar to the reference sleep state, in some embodiments, the second sleep state will be stored as part of a larger data structure or class, and the second sleep state may be obtained through accessor methods. In some embodiments, the second sleep state may be converted from a string or other non-numeric data type into a numeric data type by means of an appropriate hashing algorithm, or the second sleep state may be a primitive data structure, and may be directly used in the comparison at line 609.

At line 809, control circuitry 304 compares the value of A to the value of B to see if they are essentially equivalent. This is achieved by subtracting the value of B from A, taking the absolute value of the difference, and then comparing the absolute value of the difference to a predetermined tolerance level. In some embodiments, the tolerance level may be a set percentage of either A or B. In some embodiments, the tolerance level may be a fixed number. For example, setting the tolerance level to a set multiple of machine epsilon may allow for the algorithm to account for small rounding errors that may result from the use of floating point arithmetic. In some embodiments, the tolerance level may be set to zero, or the condition inside the IF statement may be replaced with a strict equivalence between A and B.

At line 810, control circuitry 304 executes a subroutine to return an indication that the user is ready to be awakened, using processing circuitry, if the condition in line 809 is satisfied. In some embodiments, this may be achieved by processing circuitry 306 sending the appropriate signals to control circuitry 304. At line 811, control circuitry 304 jumps to the termination subroutine and prepares to end the process.

At line 812, control circuitry 304 executes a subroutine to indicate that the user is not yet ready to be awakened, using processing circuitry. Because the second sleep state did not match any of the reference sleep states indicative of a reference user ready to be awakened, the user is likely not yet ready to be awakened.

At line 814, control circuitry 304 runs a termination subroutine after the algorithm has performed its function. For example, in some embodiments, control circuitry 304 may destruct variables, perform garbage collection, free memory or clear the cache of processing circuitry 306.

It will be evident to one skilled in the art that the process described by the pseudocode in FIG. 8 may be implemented in any number of programming languages and a variety of different hardware, and the particular choice and location of primitive functions, logical evaluations, and function evaluations are not intended to be limiting. It will also be evident that the code may be refactored or rewritten to manipulate the order of the various logical evaluations, perform several iterations in parallel rather than in a single iterative loop, or to otherwise manipulate and optimize run-time and performance metrics without fundamentally changing the inputs or final outputs. For example, in some embodiments break conditions may be placed after lines 810 to speed operation, or the conditional statements may be replaced with a case-switch. In some embodiments, rather than iterating over all instances of reference sleep states at step 806, in some embodiments, the code may be rewritten so control circuitry 304 is instructed to evaluate multiple 10, instances of reference sleep states simultaneously on a plurality of processors or processor threads, lowering the number of iterations needed and potentially speeding up computation time.

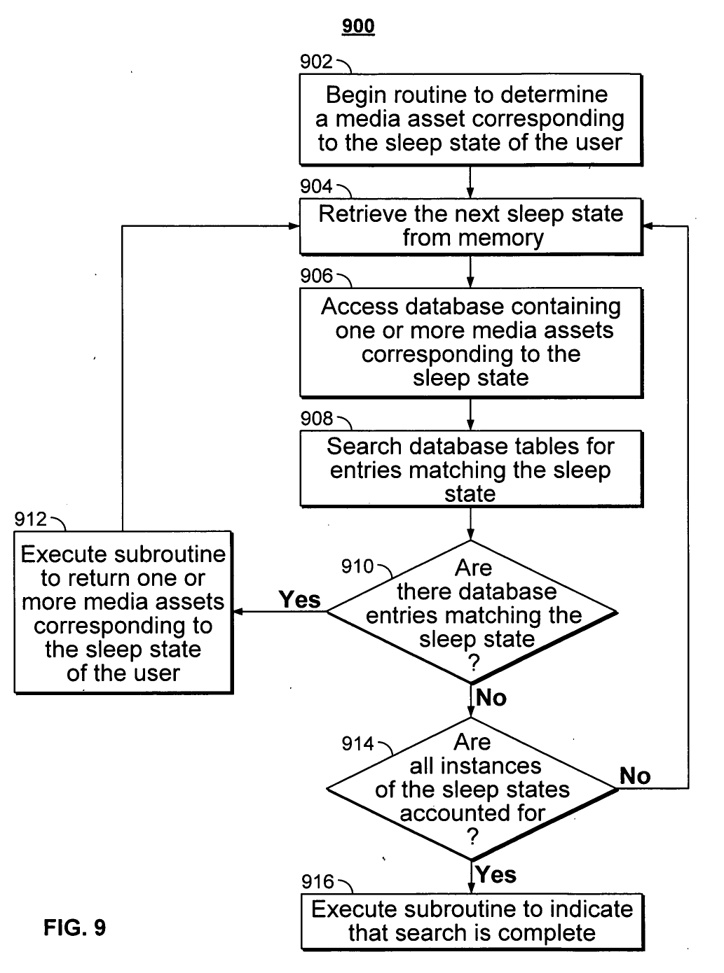

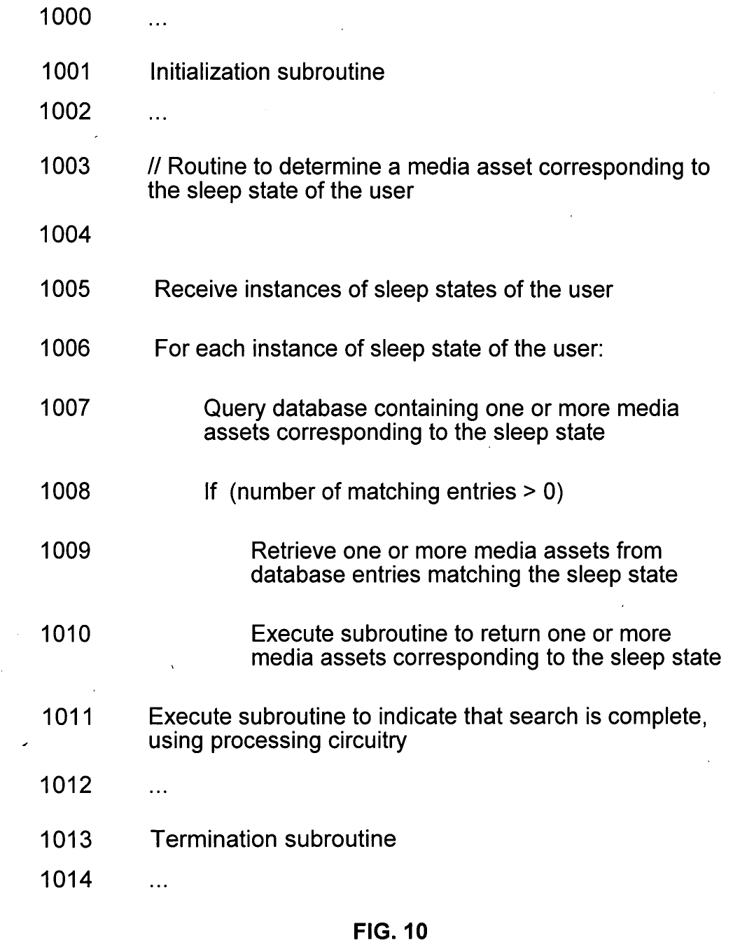

FIGS. 9 and 10 present processes implemented on control circuitry (for example, control circuitry 304) to determine a media asset corresponding to one or more sleep states of the user in accordance with some embodiments of the disclosure. Similar to the algorithms described by FIGS. 5-8, in some embodiments, this process may be encoded on to non-transitory storage medium (for example, storage device 308) as a set of instructions to be decoded and executed by processing circuitry (for example, processing circuitry 306). Processing circuitry may in turn provide instructions to other sub-circuits contained within control circuitry 304, such as the tuning, video generating, encoding, decoding, encrypting, decrypting, scaling, analog/digital conversion circuitry, and the like.

The flowchart in FIG. 9 describes a process implemented on control circuitry (for example, control circuitry 304) to search a database and determine a media asset corresponding to one or more sleep states of the user in accordance with some embodiments of the disclosure. At step 902, control circuitry 304 begins a routine to search a database and determine a media asset corresponding to one or more sleep states of the user. In some embodiments, this may be done either directly or indirectly in response to a user action or input (for example, from signals received by control circuitry 304 or user input interface 310).

At step 904, control circuitry 304 proceeds to retrieve the next sleep state from memory (for example, storage device 308). In some embodiments, control circuitry 304 may retrieve a single primitive data structure that represents the next sleep state of the user. In some embodiments, control circuitry 304 may retrieve the next sleep state from a larger class or data structure.

At step 906, control circuitry 304 accesses a database containing one or more media assets corresponding to sleep states. In some embodiments, this database may be stored locally (for example, on storage device 308) prior to beginning the algorithm. In some embodiments, the database may also be accessed by using communications circuitry to transmit information across a communications network (for example, communications network 414) to a database implemented on a remote storage device (for example, reference sleep state source 424).

At step 908, control circuitry 304 searches database tables for entries matching the sleep state of the user. In some embodiments, this may be done by comparing an identifier, for example, a string or integer representing the sleep state that matches the types of identifiers used inside the database. In some embodiments, control circuitry 304 may submit a general query to the database for table entries matching the sleep state. Control circuitry 304 may receive a list of indices or a data structure containing a portion of the database contents. In some embodiments, the database may implement a junction table that in turn cross-references entries from other databases. In this case, control circuitry 304 may retrieve indices from a first database that in turn can be used to retrieve information from a second database. Although we may describe control circuitry 304 interacting with a single database for purposes of clarity, it is understood that the algorithm of FIG. 9 may be implemented using multiple independent or cross-referenced databases.

At step 910, control circuitry 304 determines if there are database entries matching the sleep state. In some embodiments, control circuitry 304 may receive a signal from the database indicating that there are no matching entries. In some embodiments, control circuitry 304 may instead receive a list of indices or data structures with a NULL or dummy value. If control circuitry 304 identifies that there are database entries matching the sleep state, the algorithm proceeds to step 912. Otherwise, if the condition is not satisfied, the algorithm proceeds to step 914.

At step 912, control circuitry 304 executes a subroutine to return one or more media assets corresponding to the sleep state of the user. Afterwards, the algorithm may proceed to step 914 where it is determined if there are further instances of sleep states that need to be accounted for.

At step 914, control circuitry 304 determines if all instances of sleep states are accounted for and if further iterations are needed. If further iterations are needed the algorithm will loop back to step 904 where control circuitry 304 will retrieve the next instance of sleep state from memory. If no further iterations are needed the algorithm will proceed to step 916. At step 916, control circuitry 304 executes a subroutine to indicate the search is complete.

It is contemplated that the descriptions of FIG. 9 may be used with any other embodiment of this disclosure. In addition, the descriptions described in relation to process 900 of FIG. 9 may be done in alternative orders or in parallel to further the purposes of this disclosure. For example, control circuitry 304 may submit multiple queries to the database in parallel, or it may submit multiple queries to a plurality of similar databases in order to reduce lag and speed the execution of process 900. Furthermore, it should be noted that the process of FIG. 9 may be implemented on a combination of appropriately configured software and hardware, and that any of the devices or equipment discussed in relation to FIGS. 3-4 could be used to implement one or more portions of the algorithm.

The pseudocode in FIG. 10 describes a process to determine, using a database, a media asset corresponding to one or more sleep states of the user in accordance with some embodiments of the disclosure. It will be evident to one skilled in the art that the process described by the pseudocode in FIG. 10 may be implemented in any number of programming languages and a variety of different hardware, and that the style and format should not be construed as limiting, but rather a general template of the steps and procedures that would be consistent with code used to implement some embodiments of this disclosure.

At line 1001, control circuitry 304 runs a subroutine to initialize variables and prepare to determine, using a database, a media asset corresponding to one or more sleep states of the user, which begins on line 1005. For example, in some embodiments, control circuitry 304 may copy instructions from non-transitory storage medium (for example, storage device 308) into RAM or into the cache for processing circuitry 306 during the initialization stage. Additionally, in some embodiments, additional information relating to the subroutine may be retrieved, set, and stored at 1001.

At line 1005, control circuitry 304 receives instances of sleep states of the user from memory. Control circuitry 304 may retrieve the sleep states by receiving, for example, a pointer to an array of sleep states of the user. In another example, control circuitry 304 may receive an object of a class, such as an iterator object containing sleep states of the user.

At line 1006, control circuitry 304 iterates through the instances of sleep states of the user. If only a single sleep state is indicated, the loop will only execute once. This loop may be implemented in multiple fashions depending on the choice of hardware and software language used to implement the algorithm of FIG. 10; for example, this may be implemented as part of a “for” or “while” loop.

At line 1007, control circuitry 304 accesses a database containing one or more media assets corresponding to the sleep state. In some embodiments, this database may be stored locally (for example, on storage device 308) prior to beginning the algorithm. In some embodiments, the database may also be accessed by using communications circuitry to transmit information across a communications network (for example, communications network 414) to a database implemented on a remote storage device (for example, reference sleep state source 424).

Control circuitry 304 searches database tables for entries matching the sleep state. In some embodiments, this may be done by comparing an identifier, for example, a string or integer representing the sleep state that matches the types of identifiers used inside the database. In some embodiments, control circuitry 304 may submit a general query to the database for table entries matching the sleep state. Control circuitry 304 may receive a list of indices or a data structure containing a portion of the database contents. In some embodiments, the database may implement a junction table that in turn cross-references entries from other databases. In this case, control circuitry 304 may retrieve indices from a first database that in turn can be used to retrieve information from a second database. Although we may describe control circuitry 304 interacting with a single database for purposes of clarity, it is understood that the algorithm of FIG. 10 may be implemented using multiple independent or cross-referenced databases.

At line 1008, control circuitry 304 determines if any database entries match the sleep state. In some embodiments, control circuitry 304 may receive a signal from the database indicating that there are no matching entries. In some embodiments, control circuitry 304 may instead receive a list of indices or data structures with a NULL or dummy value. If control circuitry 304 identifies that there are database entries matching the sleep state, control circuitry 304 proceeds to line 1009.

If the condition in line 1008 is satisfied, at line 1009, control circuitry 304 retrieves one or more media assets from the database entries matching the sleep state. At line 1010, control circuitry 304 executes a subroutine to return one or more media assets corresponding to the sleep state. In some embodiments, this may be achieved by processing circuitry 306 sending the appropriate signals to suitable circuitry.

At line 1011, control circuitry 304 executes a subroutine to indicate that the search is complete. At line 1013, control circuitry 304 runs a termination subroutine after the algorithm has performed its function. For example, in some embodiments, control circuitry 304 may destruct variables, perform garbage collection, free memory or clear the cache of processing circuitry 306.

It will be evident to one skilled in the art that the process described by the pseudocode in FIG. 10 may be implemented in any number of programming languages and a variety of different hardware, and the particular choice and location of primitive functions, logical evaluations, and function evaluations are not intended to be limiting. It will also be evident that the code may be refactored or rewritten to manipulate the order of the various logical evaluations, perform several iterations in parallel rather than in a single iterative loop, or to otherwise manipulate and optimize run-time and performance metrics without fundamentally changing the inputs or final outputs. For example, in some embodiments the code may be re-written so control circuitry 304 is instructed to evaluate multiple instances of sleep states and submit multiple database queries simultaneously using a plurality of processors or processor threads.

It is also understood that although we may describe control circuitry 304 interacting with a single database, this is only a single embodiment described for illustrative purposes, and the algorithm of FIG. 10 may be implemented using multiple independent or cross-referenced databases. For example, a database stored locally (for example, on storage 308) may index or cross-reference a database stored remotely (for example, media guidance data source 418 or reference sleep state source 424), which may be accessible through any number of communication channels (for example, communications network 414). In some embodiments, this may allow control circuitry 304 to utilize a look-up table or database front-end efficiently stored on a small local drive to access a larger database stored on a remote server on demand.

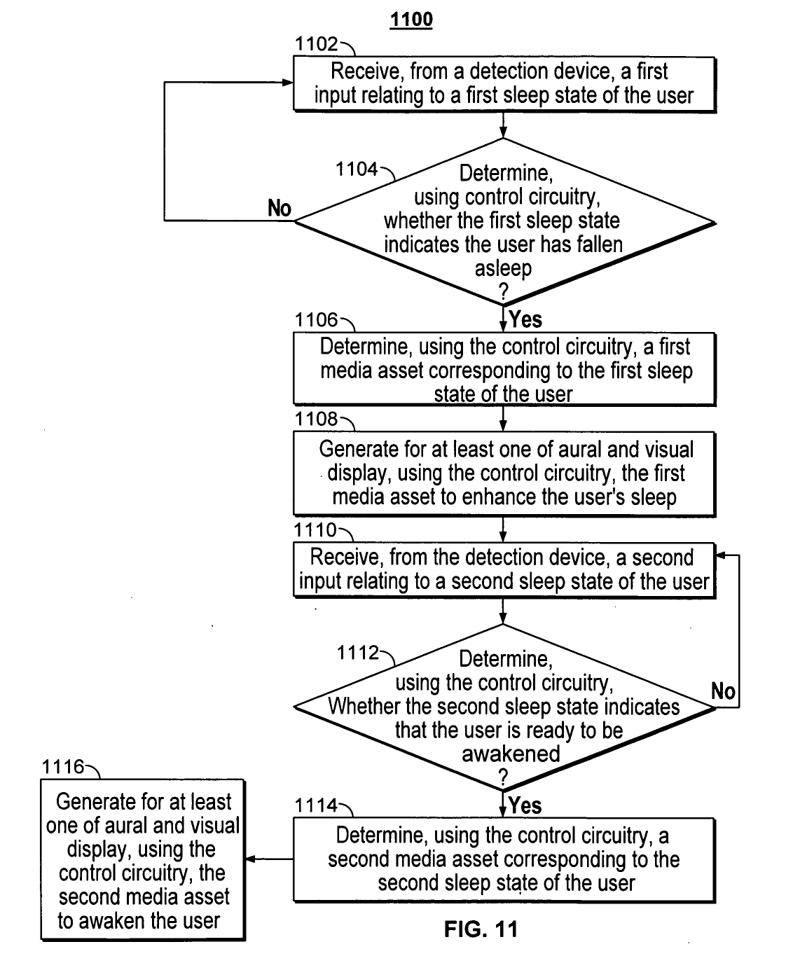

FIG. 11 is a flowchart of illustrative process 1100 for enhancing sleep of a user of an interactive media guidance system in accordance with some embodiments of the disclosure. It should be noted that process 1100 or any step thereof could be performed on, or provided by, any of the devices shown in FIGS. 3-4. For example, process 1100 may be executed by control circuitry 304 (FIG. 3), control circuitry of media content source 416, media guidance data source 418, reference sleep state source 424 (FIG. 4), or other suitable circuitry. In addition, one or more steps of process 1100 may be incorporated into or combined with one or more steps of any other process or embodiment (for example, FIGS. 5-10).

At step 1102, control circuitry 304 receives, from a detection device, a first input relating to a first sleep state of the user. For example, the first sleep state may include measurements from one or more detection devices. In some embodiments, the detection device includes one or more of a video camera, a microphone, a biometric sensor, and another suitable detection device. For example, control circuitry 304 may receive data from one or more detection devices to gather relevant information for the user and his or her sleep state.

In some embodiments, the first sleep state and the second sleep state comprise measurements of one or more of heartbeat, blood pressure, skin moisture, brain activity, rapid eye movement, and other suitable measurements. For example, control circuitry 304 may receive a position of the user from a camera (for example, sitting up indicating awake or lying down indicating asleep), a sound from or near the user from a microphone (for example, cheering sounds indicating awake or light snoring indicating asleep), a heart rate from a biometric sensor on the user (for example, a higher heart rate indicating awake or a lower heart rate indicating asleep), or another suitable measurement relating to the user's sleep state. One or more of these measurements may be included in a data structure representing the user's sleep state.

At step 1104, control circuitry 304 determines whether the first sleep state indicates that the user has fallen asleep. For example, the first sleep state may include measurements relating to the user that indicate the user has fallen asleep, such as the user may be lying down, lightly snoring, and/or have a lower heart rate. In some embodiments, control circuitry 304 determines whether the first sleep state indicates the user has fallen asleep by comparing the first sleep state to a first reference sleep state indicative of a reference user who has fallen asleep and determining whether the first sleep state substantially matches the first reference sleep state. For example, measurements from one or more detection devices may be included in a data structure representing the first sleep state. Control circuitry 304 may compare the first sleep state to the reference sleep state and determine whether the corresponding measurements differ within or beyond a threshold. If the condition is satisfied, control circuitry 304 proceeds to step 1106; otherwise, control circuitry 304 loops back to step 1102.

At step 1106, control circuitry 304 determines a first media asset corresponding to the first sleep state of the user. The first media asset enhances the user's sleep. For example, control circuitry 304 may select a media asset including soothing sounds and light to help the user fall into deep sleep. In another example, control circuitry 304 may select a media asset for an audio foreign language lesson to help the user learn the foreign language. In some embodiments, control circuitry 304 determines the first media asset corresponding to the first sleep state of the user by retrieving, from memory, one or more media assets specified by the user for enhancing the user's sleep and selecting the first media asset from the one or more media assets. For example, the user may have selected an audio foreign language lesson to be played back when the user falls asleep. In some embodiments, control circuitry 304 determines the first media asset corresponding to the first sleep state of the user by retrieving, from memory, a user profile for the user, searching a database for one or more media assets for enhancing the user's sleep based on the user profile, and automatically selecting the first media asset from the one or more media assets. For example, the user may have been listening to an audio foreign language lesson when he or she fell asleep. Control circuitry 304 may select the next audio lesson in the series when the user falls asleep to help the user learn the foreign language.

In some embodiments, the first media asset and the second media asset comprise one of an audio asset, a video asset, and an audiovisual asset. For example, control circuitry 304 may select an audio asset including drumming music, an audio foreign language lesson, a media asset including soft green light, a media asset including soothing sounds and light, or another suitable media asset. At step 1108, control circuitry 304 generates for aural or visual display the first media asset to enhance the user's sleep.

At step 1110, control circuitry 304 receives, from the detection device, a second input relating to a second sleep state of the user. For example, the second sleep state may include measurements from one or more detection devices, such as a video camera, a microphone, and a biometric sensor. At step 1112, control circuitry 304 determines whether the second sleep state indicates that the user is ready to be awakened. For example, the second sleep state may include measurements relating to the user that indicate the user is ready to be awakened, such as the user may be moving while sleeping, not snoring, and/or have a higher heart rate. In some embodiments, control circuitry 304 determines whether the second sleep state indicates the user is ready to be awakened by comparing the first sleep state to a second reference sleep state indicative of a reference user who is ready to be awakened and determining whether the first sleep state substantially matches the second reference sleep state. For example, measurements from one or more detection devices may be included in a data structure representing the second sleep state. Control circuitry 304 may compare the second sleep state to the reference sleep state and determine whether the corresponding measurements differ within or beyond a threshold. In another example, control circuitry 304 may compare corresponding measurements from the received sleep state and reference sleep state, e.g., whether the user's current heartbeat of 52 beats per minute differs, within or beyond a threshold, from the reference heartbeat of 60 beats per minute, as well as check other conditions, for example, the user requested to be awakened if he is asleep at 4:30 PM. In this example, comparing the sleep states requires two comparisons, one comparison for a heartbeat of less than 60 beats per minute and another comparison to find out if the time of day is before 4:30 PM. If the condition is satisfied, control circuitry 304 proceeds to step 1114; otherwise, control circuitry 304 loops back to step 1110.

At step 1114, control circuitry 304 determines a second media asset corresponding to the second sleep state of the user. The second media asset wakes up the user. For example, control circuitry 304 may select a media asset including gradually increasing sounds and light to help the user awaken. In another example, control circuitry 304 may stop playback of a media asset for an audio foreign language lesson to help the user awaken. In some embodiments, control circuitry 304 determines the second media asset corresponding to the second sleep state of the user by retrieving, from memory, one or more media assets specified by the user for waking up the user and selecting the second media asset from the one or more media assets. For example, control circuitry 304 may select a media asset including gradually increasing sounds and light to help the user awaken. In another example, control circuitry 304 may select no media asset or a blank media asset, for example, to stop playback of a media asset for an audio foreign language lesson, to help the user awaken. In some embodiments, control circuitry 304 determines the second media asset corresponding to the second sleep state of the user by retrieving, from memory, a user profile for the user, searching a database for one or more media assets for waking up the user based on the user profile, and automatically selecting the second media asset from the one or more media assets. For example, control circuitry 304 may select a media asset including drumming sounds based on the user profile indicating that the user likes waking up to such sounds. At step 1116, control circuitry 304 generates for aural or visual display the second media asset to awaken the user.

It is contemplated that the steps or descriptions of FIG. 11 may be used with any other embodiment of this disclosure. In addition, the steps and descriptions described in relation to FIG. 11 may be done in alternative orders or in parallel to further the purposes of this disclosure. For example, each of these steps may be performed in any order or in parallel or substantially simultaneously to reduce lag or increase the speed of the system or method. Furthermore, it should be noted that any of the devices or equipment discussed in relation to FIGS. 3-4 could be used to perform one or more of the steps in FIG. 11.

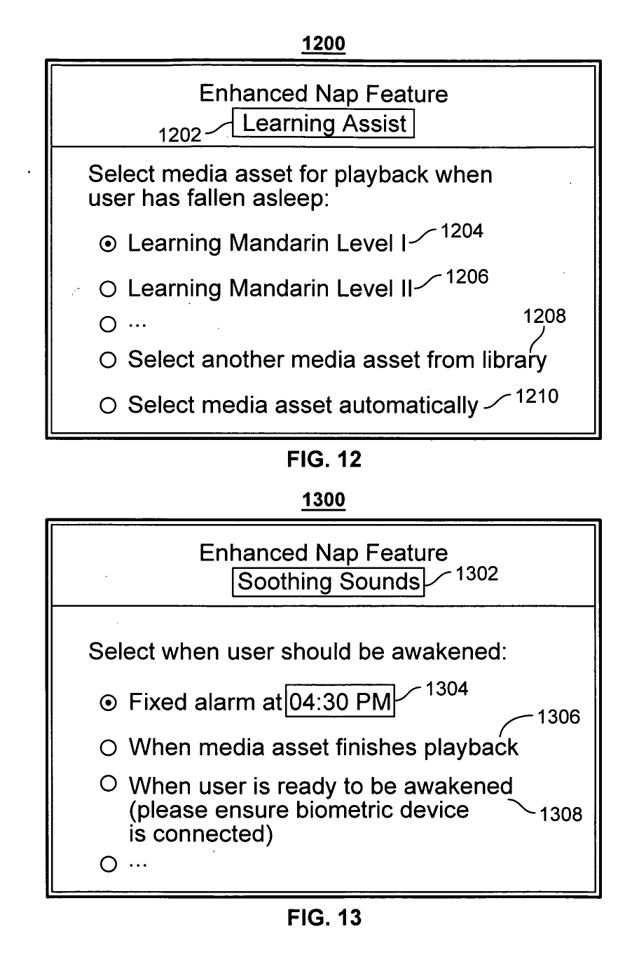

FIGS. 12 and 13 are diagrams of illustrative displays relating to enhancing sleep of a user of an interactive media guidance system in accordance with some embodiments of the disclosure. Sound and light, alone or in combination, can alter people's sleep states and enhance the sleeping experience when they nap. In some embodiments, the interactive media guidance application, implemented on control circuitry (for example, control circuitry 304), detects the user has fallen asleep based on the user's sleep state. For example, when the interactive media guidance application detects the user has fallen asleep, the interactive media guidance application may turn off the television display.

In another example, the interactive media guidance application, implemented on control circuitry (for example, control circuitry 304), may instruct a speaker to produce certain sounds, such as drumming music, to help soothe the user during sleep. In yet another example, the interactive media guidance application may instruct a display to produce certain types of light, such as soft green light, to help soothe the user during sleep. In yet another example, the interactive media guidance application may initiate subliminal learning by playing an audio lesson for a foreign language the user wishes to learn.

Additionally and optionally, the interactive media guidance application, implemented on control circuitry (for example, control circuitry 304), may select specific information to be conveyed during specific sleep states that are conducive to learning. It is well known that different states of consciousness associated with the various sleep states have varying degrees of susceptibility in human beings. The interactive media guidance application may promote the desired information personalized to the user as he or she is going into a napping state to promote the desired effect or communicate the desired information.

In one example, the system provides a user an option for alert dozing which can be accompanied by educational audio and/or images. Language-learning while dozing is a well-known example of this learning technique. FIG. 12 provides an illustrative example of such a learning assist feature 1202. In the illustrative example, Albert makes the most of his habit of drifting off to sleep around 4:00 PM on Saturday and Sunday afternoons in front of his television display. In display 1200, Albert selects option 1204 to help him learn Level I of spoken Mandarin Chinese. Alternatively, Albert may select option 1206 for Level II of learning spoken Mandarin Chinese, option 1208 for another media asset from his library, option 1210 for selection of the media asset automatically (for example, based on past selection history), or another suitable option.

Using detection devices, including a camera and biometric sensors, to provide heartbeat, blood pressure, skin moisture readings, and rapid eye movement, the interactive media guidance application, implemented on control circuitry (for example, control circuitry 304), detects when Albert falls asleep. The interactive media guidance application plays audio conversations in Mandarin Chinese at Albert's appropriate difficulty level for 20 minutes. After 20 minutes, the interactive media guidance application raises the audio speaker volume and creates a buzzing sound accompanied by flashing bright colors on the television display to wake Albert. Albert sometimes also uses an EEG headband that provides the interactive media guidance application data regarding Albert's sleep levels. In such cases, the interactive media guidance application uses the EEG headband data to determine when to wake Albert to avoid grogginess due to sleep inertia.

In another example, the interactive media guidance application, implemented on control circuitry (for example, control circuitry 304), provides a user an option for deep sleep in which the system turns off all sound and darkens its display. The interactive media guidance application may turn on a noise cancellation feature if the user is wearing earphones or headphones or another suitable headset. The user may vary his or her nap profile according to the time of day, the day of the week, or other factors. The system may automatically initiate a set of sleep enhancing features at midnight while initiating a different set of sleep enhancing features just after lunchtime.

In some embodiments, the interactive media guidance application, implemented on control circuitry (for example, control circuitry 304), automatically sets an alarm to awaken the user after a specific length of time. FIG. 13 provides an illustrative example of such an alarm feature. In display 1300, the user selects Soothing Sounds feature 1302 to play one or more sounds that may help the user fall asleep. For example, the user may choose to take a nap and doze with soothing sounds and light or be escorted into a more alert state and have a French lesson played during his or her nap for the desired outcome of the user becoming more proficient in the French language. The user may select option 1304 which automatically sets to awaken the user after a half-hour nap at 4:30 PM. Alternatively, the user may set how long his or her nap should be based on the time, day, or other conditions when he or she falls asleep.

The user may select option 1306 to be awakened when a selected media asset, such as the French lesson, finishes playback. The user may select option 1308 to be awakened when the interactive media guidance application detects the user is ready to be awakened. The interactive media guidance application may use an electrical signal, for example, generated via a smart watch worn by the user, to gently wake the user and avoid grogginess due to sleep inertia. In some embodiments, the interactive media guidance application detects the user is ready to be awakened based on the user's sleep state. The interactive media guidance application may wake the user at the proper time for the desired state of consciousness. For example, the interactive media guidance application may detect the user's brain activity and avoid waking the user during slow-wave sleep to avoid grogginess due to sleep inertia. The interactive media guidance application may additionally and optionally provide selectable options to the user to wake to an audio or video asset, an audio alert, an electrical signal from a biometric device worn by the user, or another suitable alert.

In some embodiments, the interactive media guidance application, implemented on control circuitry (for example, control circuitry 304), allows the user to catch up on media assets the user missed while he or she was sleeping. The interactive media guidance application may detect the user has fallen asleep and initiate recording of the media asset currently being viewed. When the interactive media guidance application wakes up the user, the interactive media guidance application may offer selectable options to provide a summary of the portion of the media asset the user missed, to play back the recording of the portion of the media asset the user missed, or to play back only important scenes from the portion of the media asset the user missed. The interactive media guidance application may set an alarm for an important scene that the user may not want to miss. Additionally and optionally, the interactive media guidance application may allow the user to choose how long he or she should be allowed to nap before he or she is awakened. The interactive media guidance application may set an automatic alarm to awaken the user accordingly.

The above-described embodiments of the present disclosure are presented for purposes of illustration and not of limitation, and the present disclosure is limited only by the claims that follow. Furthermore, it should be noted that the features and limitations described in any one embodiment may be applied to any other embodiment herein, and flowcharts or examples relating to one embodiment may be combined with any other embodiment in a suitable manner, done in different orders, or done in parallel. In addition, the systems and methods described herein may be performed in real time. It should also be noted, the systems and/or methods described above may be applied to, or used in accordance with, other systems and/or methods.

What is claimed is:

1. A method for enhancing sleep of a user implemented in an interactive media guidance application, comprising:
receiving, from a detection device, a first input relating to a first sleep state of the user;
determining, using control circuitry, whether the first sleep state indicates that the user has fallen asleep;
in response to determining that the first sleep state indicates that the user has fallen asleep:
determining, using the control circuitry, a first media asset corresponding to the first sleep state of the user, wherein the first media asset enhances the user's sleep;
generating for at least one of aural and visual display, using the control circuitry, the first media asset to enhance the user's sleep;
receiving, from the detection device, a second input relating to a second sleep state of the user;
determining, using the control circuitry, whether the second sleep state indicates that the user is ready to be awakened;
in response to determining that the second sleep state indicates that the user is ready to be awakened:
determining, using the control circuitry, a second media asset corresponding to the second sleep state of the user, wherein the second media asset wakes up the user;
generating for at least one of aural and visual display, using the control circuitry, the second media asset to awaken the user.

2. The method of claim 1, wherein the detection device includes one or more of a video camera, a microphone, and a biometric sensor.

3. The method of claim 1, wherein the first sleep state and the second sleep state comprise measurements of one or more of heartbeat, blood pressure, skin moisture, and brain activity.

4. The method of claim 1, wherein determining, using the control circuitry, whether the first sleep state indicates the user has fallen asleep comprises:
comparing, using the control circuitry, the first sleep state to a first reference sleep state indicative of a reference user who has fallen asleep;
determining, using the control circuitry, whether the first sleep state substantially matches the first reference sleep state.

5. The method of claim 1, wherein determining, using the control circuitry, the first media asset corresponding to the first sleep state of the user comprises:
retrieving, from memory, one or more media assets specified by the user for enhancing the user's sleep;
selecting, using the control circuitry, the first media asset from the one or more media assets.

6. The method of claim 1, wherein determining, using the control circuitry, the first media asset corresponding to the first sleep state of the user comprises:
retrieving, from memory, a user profile for the user;
searching a database for one or more media assets for enhancing the user's sleep based on the user profile;
automatically selecting, using the control circuitry, the first media asset from the one or more media assets.

7. The method of claim 1, wherein the first media asset and the second media asset comprise one of an audio asset, a video asset, and an audiovisual asset.

8. The method of claim 1, wherein determining, using the control circuitry, whether the second sleep state indicates the user is ready to be awakened comprises:
comparing, using the control circuitry, the first sleep state to a second reference sleep state indicative of a reference user who is ready to be awakened;
determining, using the control circuitry, whether the first sleep state substantially matches the second reference sleep state.

9. The method of claim 1, wherein determining, using the control circuitry, the second media asset corresponding to the second sleep state of the user comprises:
retrieving, from memory, one or more media assets specified by the user for waking up the user;
selecting, using the control circuitry, the second media asset from the one or more media assets.

10. The method of claim 1, wherein determining, using the control circuitry, the second media asset corresponding to the second sleep state of the user comprises:
retrieving, from memory, a user profile for the user;
searching a database for one or more media assets for waking up the user based on the user profile;
automatically selecting, using the control circuitry, the second media asset from the one or more media assets.

11. A system for enhancing sleep of a user implemented in an interactive media guidance application, comprising:
a detection device configured to:
receive a first input relating to a first sleep state of the user;
a second input relating to a second sleep state of the user;
control circuitry in communication with the detection device, the control circuitry configured to:
receive, from the detection device, the first input relating to the first sleep state of the user;
determine whether the first sleep state indicates that the user has fallen asleep;
in response to determining that the first sleep state indicates that the user has fallen asleep:
determine a first media asset corresponding to the first sleep state of the user, wherein the first media asset enhances the user's sleep;
generate for at least one of aural and visual display the first media asset to enhance the user's sleep;
receive, from the detection device, the second input relating to the second sleep state of the user;
determine whether the second sleep state indicates that the user is ready to be awakened;
in response to determining that the second sleep state indicates that the user is ready to be awakened:
determine a second media asset corresponding to the second sleep state of the user, wherein the second media asset wakes up the user;
generate for at least one of aural and visual display the second media asset to awaken the user.

12. The system of claim 11, wherein the detection device includes one or more of a video camera, a microphone, and a biometric sensor.

13. The system of claim 11, wherein the first sleep state and the second sleep state comprise measurements of one or more of heartbeat, blood pressure, skin moisture, and brain activity.

14. The system of claim 11, wherein the control circuitry configured to determine whether the first sleep state indicates the user has fallen asleep comprises control circuitry configured to:
compare the first sleep state to a first reference sleep state indicative of a reference user who has fallen asleep;
determine whether the first sleep state substantially matches the first reference sleep state.

15. The system of claim 11, wherein control the circuitry configured to determine the first media asset corresponding to the first sleep state of the user comprises control circuitry configured to:
retrieve, from memory, one or more media assets specified by the user for enhancing the user's sleep;
select the first media asset from the one or more media assets.

16. The system of claim 11, wherein the control circuitry configured to determine the first media asset corresponding to the first sleep state of the user comprises control circuitry configured to:
retrieve, from memory, a user profile for the user;
search a database for one or more media assets for enhancing the user's sleep based on the user profile;
automatically select the first media asset from the one or more media assets.

17. The system of claim 11, wherein the first media asset and the second media asset comprise one of an audio asset, a video asset, and an audiovisual asset.

18. The system of claim 11, wherein the control circuitry configured to determine whether the second sleep state indicates the user is ready to be awakened comprises control circuitry configured to:
compare the first sleep state to a second reference sleep state indicative of a reference user who is ready to be awakened;
determine whether the first sleep state substantially matches the second reference sleep state.

19. The system of claim 11, wherein the control circuitry configured to determine the second media asset corresponding to the second sleep state of the user comprises control circuitry configured to:
retrieve, from memory, one or more media assets specified by the user for waking up the user;
select the second media asset from the one or more media assets.

20. The system of claim 11, wherein the control circuitry configured to determine the second media asset corresponding to the second sleep state of the user comprises control circuitry configured to:
retrieve, from memory, a user profile for the user;
search a database for one or more media assets for waking up the user based on the user profile;
automatically select the second media asset from the one or more media assets.

21. A system for enhancing sleep of a user implemented in an interactive media guidance application, comprising:
means for receiving, from a detection device, a first input relating to a first sleep state of the user;
means for determining, using control circuitry, whether the first sleep state indicates that the user has fallen asleep;
in response to determining that the first sleep state indicates that the user has fallen asleep:
means for determining, using the control circuitry, a first media asset corresponding to the first sleep state of the user, wherein the first media asset enhances the user's sleep;
means for generating for at least one of aural and visual display, using the control circuitry, the first media asset to enhance the user's sleep;
means for receiving, from the detection device, a second input relating to a second sleep state of the user;
means for determining, using the control circuitry, whether the second sleep state indicates that the user is ready to be awakened;
in response to determining that the second sleep state indicates that the user is ready to be awakened:
means for determining, using the control circuitry, a second media asset corresponding to the second sleep state of the user, wherein the second media asset wakes up the user;
means for generating for at least one of aural and visual display, using the control circuitry, the second media asset to awaken the user.

22. The system of claim 21, wherein the detection device includes one or more of a video camera, a microphone, and a biometric sensor.

23. The system of claim 21, wherein the first sleep state and the second sleep state comprise measurements of one or more of heartbeat, blood pressure, skin moisture, and brain activity.

24. The system of claim 21, wherein the means for determining, using the control circuitry, whether the first sleep state indicates the user has fallen asleep comprises:
means for comparing, using the control circuitry, the first sleep state to a first reference sleep state indicative of a reference user who has fallen asleep;
means for determining, using the control circuitry, whether the first sleep state substantially matches the first reference sleep state.

25. The system of claim 21, wherein the means for determining, using the control circuitry, the first media asset corresponding to the first sleep state of the user comprises:
means for retrieving, from memory, one or more media assets specified by the user for enhancing the user's sleep;
means for selecting, using the control circuitry, the first media asset from the one or more media assets.

26. The system of claim 21, wherein the means for determining, using the control circuitry, the first media asset corresponding to the first sleep state of the user comprises:
means for retrieving, from memory, a user profile for the user;
means for searching a database for one or more media assets for enhancing the user's sleep based on the user profile;
means for automatically selecting, using the control circuitry, the first media asset from the one or more media assets.

27. The system of claim 21, wherein the first media asset and the second media asset comprise one of an audio asset, a video asset, and an audiovisual asset.

28. The system of claim 21, wherein the means for determining, using the control circuitry, whether the second sleep state indicates the user is ready to be awakened comprises:
means for comparing, using the control circuitry, the first sleep state to a second reference sleep state indicative of a reference user who is ready to be awakened;
means for determining, using the control circuitry, whether the first sleep state substantially matches the second reference sleep state.

29. The system of claim 21, wherein the means for determining, using the control circuitry, the second media asset corresponding to the second sleep state of the user comprises:
means for retrieving, from memory, one or more media assets specified by the user for waking up the user;
means for selecting, using the control circuitry, the second media asset from the one or more media assets.

30. The system of claim 21, wherein the means for determining, using the control circuitry, the second media asset corresponding to the second sleep state of the user comprises:
means for retrieving, from memory, a user profile for the user;
means for searching a database for one or more media assets for waking up the user based on the user profile;
means for automatically selecting, using the control circuitry, the second media asset from the one or more media assets.

31. A method for enhancing sleep of a user implemented in an interactive media guidance application, comprising:
receiving, from a detection device, a first input relating to a first sleep state of the user;
determining, using control circuitry, whether the first sleep state indicates that the user has fallen asleep;
in response to determining that the first sleep state indicates that the user has fallen asleep:
determining, using the control circuitry, a first media asset corresponding to the first sleep state of the user, wherein the first media asset enhances the user's sleep;
generating for at least one of aural and visual display, using the control circuitry, the first media asset to enhance the user's sleep;
receiving, from the detection device, a second input relating to a second sleep state of the user;
determining, using the control circuitry, whether the second sleep state indicates that the user is ready to be awakened;
in response to determining that the second sleep state indicates that the user is ready to be awakened:
determining, using the control circuitry, a second media asset corresponding to the second sleep state of the user, wherein the second media asset wakes up the user;
generating for at least one of aural and visual display, using the control circuitry, the second media asset to awaken the user.

32. The method of claim 31, wherein the detection device includes one or more of a video camera, a microphone, and a biometric sensor.

33. The method of claim 31 or 32, wherein the first sleep state and the second sleep state comprise measurements of one or more of heartbeat, blood pressure, skin moisture, and brain activity.

34. The method of any one of claims 31-33, wherein determining, using the control circuitry, whether the first sleep state indicates the user has fallen asleep comprises:
comparing, using the control circuitry, the first sleep state to a first reference sleep state indicative of a reference user who has fallen asleep;
determining, using the control circuitry, whether the first sleep state substantially matches the first reference sleep state.

35. The method of any one of claims 31-34, wherein determining, using the control circuitry, the first media asset corresponding to the first sleep state of the user comprises:
retrieving, from memory, one or more media assets specified by the user for enhancing the user's sleep;
selecting, using the control circuitry, the first media asset from the one or more media assets.

36. The method of any one of claims 31-35, wherein determining, using the control circuitry, the first media asset corresponding to the first sleep state of the user comprises:
retrieving, from memory, a user profile for the user;
searching a database for one or more media assets for enhancing the user's sleep based on the user profile;
automatically selecting, using the control circuitry, the first media asset from the one or more media assets.

37. The method of any one of claims 31-36, wherein the first media asset and the second media asset comprise one of an audio asset, a video asset, and an audiovisual asset.

38. The method of any one of claims 31-37, wherein determining, using the control circuitry, whether the second sleep state indicates the user is ready to be awakened comprises:
comparing, using the control circuitry, the first sleep state to a second reference sleep state indicative of a reference user who is ready to be awakened;
determining, using the control circuitry, whether the first sleep state substantially matches the second reference sleep state.

39. The method of any one of claims 31-38, wherein determining, using the control circuitry, the second media asset corresponding to the second sleep state of the user comprises:
retrieving, from memory, one or more media assets specified by the user for waking up the user;
selecting, using the control circuitry, the second media asset from the one or more media assets.

40. The method of any one of claims 31-39, wherein determining, using the control circuitry, the second media asset corresponding to the second sleep state of the user comprises:
retrieving, from memory, a user profile for the user;
searching a database for one or more media assets for waking up the user based on the user profile;
automatically selecting, using the control circuitry, the second media asset from the one or more media assets.

41. A non-transitory computer-readable medium having instructions recorded thereon for enhancing sleep of a user implemented in an interactive media guidance application, comprising:
an instruction for receiving, from a detection device, a first input relating to a first sleep state of the user;
an instruction for determining, using control circuitry, whether the first sleep state indicates that the user has fallen asleep;
in response to determining that the first sleep state indicates that the user has fallen asleep:
an instruction for determining, using the control circuitry, a first media asset corresponding to the first sleep state of the user, wherein the first media asset enhances the user's sleep;
an instruction for generating for at least one of aural and visual display, using the control circuitry, the first media asset to enhance the user's sleep;
an instruction for receiving, from the detection device, a second input relating to a second sleep state of the user;
an instruction for determining, using the control circuitry, whether the second sleep state indicates that the user is ready to be awakened;
in response to determining that the second sleep state indicates that the user is ready to be awakened:
an instruction for determining, using the control circuitry, a second media asset corresponding to the second sleep state of the user, wherein the second media asset wakes up the user;
an instruction for generating for at least one of aural and visual display, using the control circuitry, the second media asset to awaken the user.

42. The non-transitory computer-readable medium of claim 41, wherein the detection device includes one or more of a video camera, a microphone, and a biometric sensor.

43. The non-transitory computer-readable medium of claim 41, wherein the first sleep state and the second sleep state comprise measurements of one or more of heartbeat, blood pressure, skin moisture, and brain activity.

44. The non-transitory computer-readable medium of claim 41, wherein the instruction for determining, using the control circuitry, whether the first sleep state indicates the user has fallen asleep comprises:
an instruction for comparing, using the control circuitry, the first sleep state to a first reference sleep state indicative of a reference user who has fallen asleep;
an instruction for determining, using the control circuitry, whether the first sleep state substantially matches the first reference sleep state.

45. The non-transitory computer-readable medium of claim 41, wherein the instruction for determining, using the control circuitry, the first media asset corresponding to the first sleep state of the user comprises:
an instruction for retrieving, from memory, one or more media assets specified by the user for enhancing the user's sleep;
an instruction for selecting, using the control circuitry, the first media asset from the one or more media assets.

46. The non-transitory computer-readable medium of claim 41, wherein the instruction for determining, using the control circuitry, the first media asset corresponding to the first sleep state of the user comprises:
an instruction for retrieving, from memory, a user profile for the user;
an instruction for searching a database for one or more media assets for enhancing the user's sleep based on the user profile;
an instruction for automatically selecting, using the control circuitry, the first media asset from the one or more media assets.

47. The non-transitory computer-readable medium of claim 41, wherein the first media asset and the second media asset comprise one of an audio asset, a video asset, and an audiovisual asset.

48. The non-transitory computer-readable medium of claim 41, wherein the instruction for determining, using the control circuitry, whether the second sleep state indicates the user is ready to be awakened comprises:
an instruction for comparing, using the control circuitry, the first sleep state to a second reference sleep state indicative of a reference user who is ready to be awakened;
an instruction for determining, using the control circuitry, whether the first sleep state substantially matches the second reference sleep state.

49. The non-transitory computer-readable medium of claim 41, wherein the instruction for determining, using the control circuitry, the second media asset corresponding to the second sleep state of the user comprises:
an instruction for retrieving, from memory, one or more media assets specified by the user for waking up the user;
an instruction for selecting, using the control circuitry, the second media asset from the one or more media assets.

50. The non-transitory computer-readable medium of claim 41, wherein the instruction for determining, using the control circuitry, the second media asset corresponding to the second sleep state of the user comprises:
an instruction for retrieving, from memory, a user profile for the user;
an instruction for searching a database for one or more media assets for waking up the user based on the user profile;
an instruction for automatically selecting, using the control circuitry, the second media asset from the one or more media assets.
